(19) United States
(12) Patent Application Publication
(10) Pub. No.: US 2005/0008588 A1
(43) Pub. Date: Jan. 13, 2005

(54) Aminoarylvinyl-s-triazine compounds and uses thereof

(75) Inventors: Didier Candau, Bievres (FR); Albert Duranton, Maison Laffite (FR); Francis Pruche, Senlis (FR); Herve Richard, Villepinte (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/859,404

(22) Filed: Jun. 3, 2004

Related U.S. Application Data
(60) Provisional application No. 60/508,312, filed on Oct. 6, 2003.

(30) Foreign Application Priority Data
Jun. 5, 2003 (FR) 0306800

(51) Int. Cl. A61K 7/42; A61K 7/44

(52) U.S. Cl. 424/59

(57) ABSTRACT

The invention relates to the use of at least one agent for screening out light radiation with a wavelength ranging from 370 to 500 nm as an agent for inhibiting the degradation of endogenous carotenoids present in the skin. The invention is useful for treating skin disorders caused by sunlight (e.g., solar urticaria, actinic dermatitis and pigmentation marks). The invention also relates to novel aminoarylvinyl-s-triazine compounds and to the use thereof as agents for screening out light radiation ranging from 370 to 500 nm.

### REFERENCE TO PRIOR APPLICATIONS

This application claims priority to U.S. provisional application 60/508,312 filed Oct. 6, 2003, and to French patent application 0306800 filed Jun. 5, 2003, both incorporated herein by reference.

### FIELD OF THE INVENTION

The invention relates to novel aminoarylvinyl-s-triazine compounds and to the use thereof as agents for screening out blue light. Also described is the use of at least one agent capable of screening out light radiation with a wavelength of from 370-500 nm, for example in the manufacture of a composition applied to the surface of the skin as an agent for inhibiting the degradation of the endogenous carotenoids present in the skin. The invention further relates to cosmetic and/or dermatological compositions containing the invention aminoarylvinyl-s-triazine compounds.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The skin is subject to attack by sunlight, the consequence of which is to increase oxidative attacks, for instance:
depletion of the antioxidant capacities of the skin, lipid peroxidation and impairment of the barrier function (Thiele, J. J. Skin Pharmacol. Appl. Skin Physiol. 2001; 14 Suppl. 1: 87-91)
the oxidation of proteins (“Photoaging is associated with protein oxidation in human skin in vivo”, Sander, C. S., Chang, H., Salzmann, S., Muller, C. S., Ekanayake-Mudiyanselage, S., Elsner, P., Thiele, J. J., J. Invest. Dermatol. 2002 April; 118(4): 618-25) resulting in an impairment of the complexion of the skin.

Natural physiological protection of the skin against these attacks exists by virtue of the endogenous carotenoids present in the live layers of the skin. These carotenoids of dietary origin are also an important factor in the perception of the colour of the skin, in particular via their contribution towards the complexion (red/yellow component) (Alaluf, S., Heinrich, U., Stahl, W., Tronnier, H., Wiseman, S. “Dietary carotenoids contribute to normal human skin color and UV photosensitivity”, J. Nutr. 2002 March; 132(3): 399-403).

The cosmetic importance of protecting this reserve of endogenous carotenoids present in the skin in order to preserve the natural complexion of the skin is appreciated.

There is a need to find a means for preserving the natural complexion and the natural antioxidant protection of the skin afforded by the endogenous carotenoids present in the skin.

### DETAILED DESCRIPTION OF THE PREFERRED EMBODIMENTS

After intense research studies, the inventors have discovered that light radiation with a wavelength ranging from 370 to 500 nm (i.e., wavelengths of 370-500 nm) (corresponding to blue light) has a tendency to substantially reduce the endogenous reserve of skin carotenoids. To the inventors' knowledge, this technical relationship has never before been posed. Working from this discovery, the inventors have discovered, surprisingly and unexpectedly, a way to conserve the endogenous reserve of skin carotenoids by applying to the surface of the skin an agent for screening out light radiation with a wavelength ranging from 370 to 500 nm. This makes it possible to obtain simultaneously good protection of the natural complexion of the skin and good natural antioxidant protection of the skin.

The inventors have also discovered novel aminoarylvinyl-s-triazine compounds, the formula of which will be defined in detail later in this description, which have properties of absorbing light radiation within the wavelength range of from 370 to 500 nm.

One subject of the invention is thus the use of at least one agent for screening out light radiation with a wavelength ranging from 370 to 500 nm, in the manufacture of a composition applied to the surface of the skin as an agent for inhibiting the degradation of the endogenous carotenoids present in the skin.

Another subject of the invention is novel aminoarylvinyl-s-triazine compounds of general formula (III) below that will be defined in greater detail later in this description.

The invention also relates to compositions (e.g., cosmetic or dermatological comprising, in a physiologically acceptable medium, the aminoarylvinyl-s-triazine compounds of general formula (III).

Another subject of the invention is the use of the aminoarylvinyl-s-triazine compounds of general formula (III) in a composition as an agent for screening out light radiation in the wavelength range of from 370 to 500 nm.

The expression “agent for screening out light radiation with a wavelength of 370 to 500 nm” means any organic or mineral, synthetic or natural compound or mixture of compounds capable of screening out light radiation flaaing within the range of 370 to 500 nm and having good compatibility with the skin. Preferably the agent is capable of screening out the entire range of 370-500 nm. In this regard their absorption spectrum in this range will preferably show an absorption maximum that is between 380 and 450 nm (inclusive of endpoints). The agent may be a simple molecule or a polymer, for example. The filtration of light may result from any or all of an absorption, a reflection and/or scattering phenomenon.

Included among the agents for screening out light radiation with a wavelength of 370 to 500 nm which may be used according to the invention, special mention may be made of:
(i) flavonoid polyphenols and non-flavonoid polyphenols and also the ester, ether, heteroside and polymeric derivatives thereof;
(ii) carotenoids and β-laines;
(iii) chlorophylls;
(iv) natural or synthetic melanins;
(v) carbohydrates;
(vi) yellow or orange-yellow mineral pigments;
(vii) azo or quinone compounds;
(viii) nitrobenzene-based dyes;
(ix) aryl vinylene ketone-based compounds;
(x) aminoarylvinyl-s-triazine compounds;
and mixtures thereof.

According to one particular form of the invention, the agents for screening out light radiation with a wavelength ranging from 370 to 500 nm according to the invention will not have any antioxidant activity. In this case, they will preferably be selected from the group consisting of groups (v) to (x) above and mixtures thereof.

I Polyphenols

a) Flavonoid Polyphenols:

Included among the flavonoid polyphenols that may be used according to the invention, special mention may be made of natural yellow or orange-yellow dyes of the flavone, flavanol, isoflavone, chalcone or aurone type, as described in: “Guide des teintures naturelles. Plantes, lichen, champignons, molusques et insectes [Guide of natural dyes. Plants, lichen, fungi, molluscs and insects]”, Dominique Cardon, published by Delachaux and Niestle, ISBN 2-603-00732-7, in “Comparative Biochemistry of the Flavonoids” J. B. Harbome, Academic Press 1967, and they are especially described in the book by Harbome, J. B.: “Methods in Plant Biochemistry”, first edition, J. B. Harbome Ed.; Academic Press: London 1989.

In these dyes, the dyeing principles most commonly known are luteolin, aureusidin, bracteatin, sulfuretin, maritimein, leptosin, sulfurin, fustin, quercitin, rutin, apigenin, apioside and morin, and also all the ester, ether, heteroside and yellow polymeric derivatives thereof.

Included among the flavonoid polyphenols that may be used according to the invention, particular mention may also be made of yellow or orange-yellow proanthocyanidine oligomers or hydrolysable (gallic, ellagic) and non-hydrolysable (catechin type) condensed tannin polymers, of natural or synthetic origin, as described in: “Polyphenols: chemistry, dietary sources, metabolism and nutritional significance”, Bravo, L. Nutr. Rev. 1998 November; 56(11): 317-33.

In these dyes, the dyeing principles most commonly known are tannic acid and catechin polymers extracted from grape, tea, coffee, cocoa, chicory, onion or yellow apple peel.

b) Non-flavonoid Polyphenols

Included among the non-flavonoid polyphenols that may be used according to the invention, special mention may be made of natural or synthetic yellow or orange-yellow dyes of flavin (riboflavin, riboflavin 5′-phosphate) and curcumin type, as described in: “Guide des teintures naturelles. Plantes, lichen, champignons, molusques et insectes [Guide of natural dyes. Plants, lichen, fungi, molluscs and insects]”, Dominique Cardon, published by Delachaux and Niestle, ISBN 2-603-00732-7 and in “Natural food colorants” ed. G. A. F. Hendry & J. D. Houghton, Blackie Academic & Professional, 1996, ISBN 0 7514 0231 1.

Included among the polyphenols that will preferably be used are the polymeric forms and especially proanthocyanidin oligomers or condensed tannin polymers of hydrolysable type (gallic, ellagic) and non-hydrolysable type (catechin type), and more particularly procyanidin B2 from apple, tannic acid and catechin polymers extracted from grape, tea, coffee, cocoa, chicory, onion or yellow apple peel.

II Carotenoids and β-laines

Included among the carotenoids that may be used according to the invention, examples that may be specially mentioned include bixin, crocetin, crocin, lutein, zeoaxanthin, astaxanthin, canthaxanthin, capsanthin, cryptoxanthin, rhodoxanthin and rubixanthin.

Included among the β-laines that may be used according to the invention, special mention may be made of vulgaxanthins I and II.

III Chlorophylls

Included among the chlorophylls that may be used according to the invention, chlorophylls a, b, c and d and the acidic or alkaline hydrolysates thereof may be chosen, for example.

IV Melanins

Included among the melanins of natural or synthetic origin, which are soluble or in the form of pigments, special mention may be made of pheomelanin-enriched melanins.

V Carbohydrates

Included among the carbohydrates that may be used according to the invention, special mention may be made of the yellow-coloured products derived from the heating or oxidation of monosaccharides or polysaccharides (glucose, fructose or sucrose), such as caramel.

VI Yellow or Orange Mineral Pigments

Included among the mineral pigments that may be mentioned in particular are those of the iron oxide type and more particularly nanopigments with an elemental particle size of less than 100 nm, such as
the transparent yellow iron oxide (10 nm×100 nm needles) sold under the name “Cappoxyt jaune 4214X” by the company Capelle;
the stearine-coated yellow iron oxide (10 nm) sold under the name “transparent iron oxide” by BASF;
the micronized yellow iron oxide sold under the name “TY-220” by the company Mitsubishi.
   VII Dyes of the Azo or Quinone Type

Included among the natural or synthetic dyes of the quinone type that may be used according to the invention, examples that may be specially mentioned include those of yellow or orange-yellow naphthoquinone type and the oxidation products thereof, such as juglone and lawsone; those of anthraquinone type and the yellow or orange-yellow oxidation products thereof that have good compatibility with the skin.

Included among the dyes of the azo type that may be used according to the invention, an example that may be mentioned is tartrazine, having the structure:

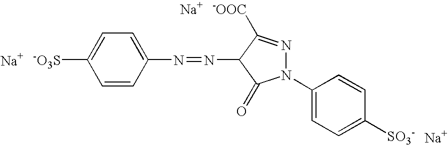

One particular family of dyes of the azo type and of the quinone type that may preferably be used according to the invention is described in patent application EP 0 839 815, of formula (1), (2) or (3): 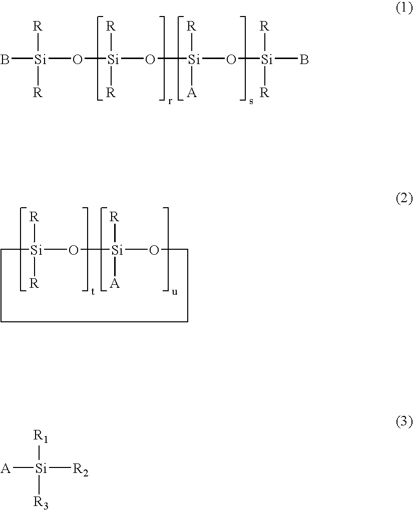
in which, for formulae (1) and (2):
R, which may be identical or different, are selected from the group consisting of C₁-C₁₀ alkyl, phenyl and 3,3,3-trifluoropropyl radicals, at least 80%, in numerical terms, of the radicals R being methyl,
B, which may be identical or different, are selected from the group consisting of the radicals R above and the radical A defined below,
r is an integer between 0 and 50 inclusive, and s is an integer between 0 and 20 inclusive, with the condition that if s is 0, then at least one of the two symbols B denotes A,
u is an integer between 1 and 6 inclusive, and
t is an integer between 0 and 10 inclusive, it being understood that t+u is greater than or equal to 3,
R₁, R₂ and R₃, which may be identical or different, are selected from the group consisting of saturated or unsaturated, linear or branched C₁-C₈ alkyl and alkenyl radicals,
the symbols A, which may be identical or different, denote a radical of formula (4a), (4b), (4c) or (4d) below: 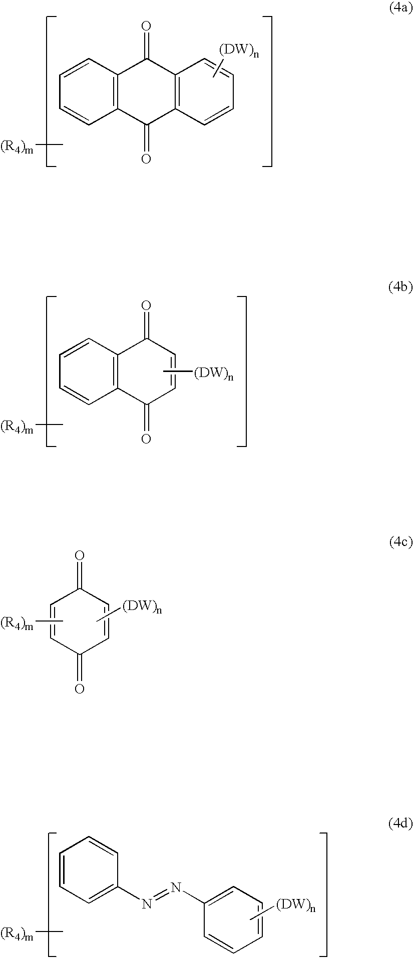
R₄, which may be identical or different, represent a linear or branched C₁-C₆ alkyl radical, an OH, C₁-C₄ alkoxy, hydroxy(C₁-C₄)alkyl, COOH, CONH₂, CN, SO₃H, halogen or NO₂ radical, a radical NR₅R₆ in which R₅ and R₆, which may be identical or different, denote a hydrogen atom or a C₁-C₈ alkyl, hydroxy(C₁-C₄)alkyl or amino(C₁-C₄)alkyl radical, or form, together with the nitrogen atom to which they are attached, a 5- or 6-membered heterocycle optionally interrupted with an oxygen or sulfur atom,
m is an integer between 0 and 2 inclusive,
n is an integer equal to 1 or 2,
D is an —SO₂NH—, —CONH— or —O— radical or a radical —NR₇— in which R₇ is H or CH₃,
W is a divalent radical of formula (5): 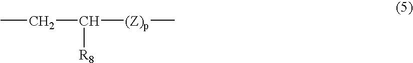
   or of formula (6):
   —HC═CH—(Z)ₚ— (6)
   in which
R₈ denotes a hydrogen atom, a hydroxyl radical or a linear or branched, saturated or unsaturated C₁-C₈ alkyl radical,
Z is a linear or branched C₁-C₆ alkylene radical optionally substituted with an OH radical or a linear or branched, saturated or unsaturated C₂-C₈ alkoxy radical,
p is an integer equal to 0 or 1.

According to the invention, the compounds of formula (1), (2) or (3) that are more particularly preferred are the following:
4-(4-dimethylaminophenylazo)-N-[3-[1,3,3,3-tetramethyl-1-[(trimethylsilyl)oxy]disiloxanyl]propyl]benzenesulfonamide [compound (7)]: 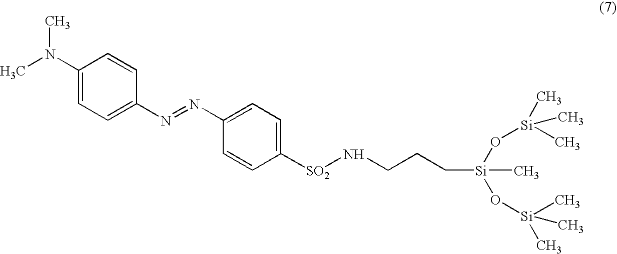
4-(4-dimethylaminophenylazo)-N-[3-[1,3,3,3-tetramethyl-1-[(trimethylsilyl)oxy]disiloxanyl]propyl]benzamide [compound (8)]: 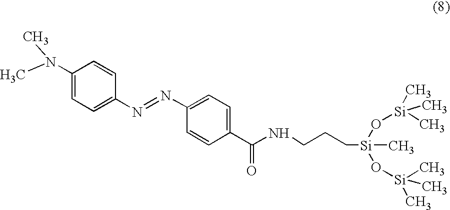
2-(4-methoxy-2-nitrophenylazo)-5-(3-trimethylsilanylpropoxy)phenol [compound (9)]: 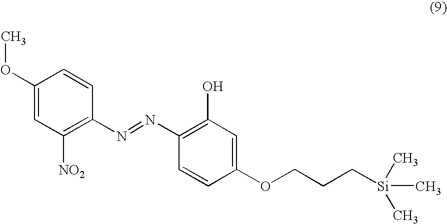
2,5-bis-[3-[1,3,3,3-tetramethyl-1-[(trimethylsilyl)oxy]disiloxanyl]-propylamino]benzoquinone [compound (10)]: 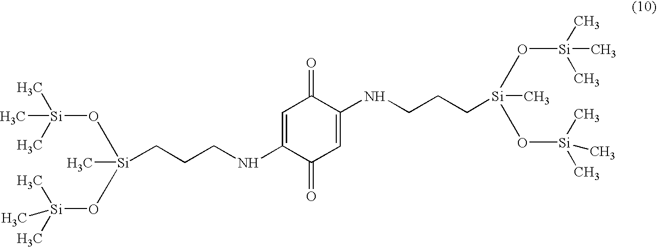
2-chloro-3-[3-[1,3,3,3-tetramethyl-1-[(trimethylsilyl)-oxy]disiloxanyl]propylamino]-[1,4]-naphthoquinone [compound (11)]: 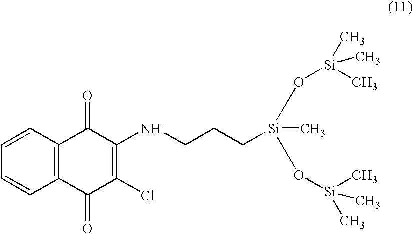
2-[3-[1,3,3,3-tetramethyl-1-[(trimethylsilyl)oxy]disiloxanyl]propylamino]-[1,4]-naphthoquinone [compound (12)]: 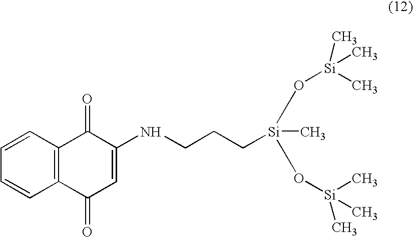
1-hydroxy-4-[3-[1,3,3,3-tetramethyl-1-[(trimethylsilyl)-oxy]disiloxanyl]propylamino]anthraquinone [compound (13)]: 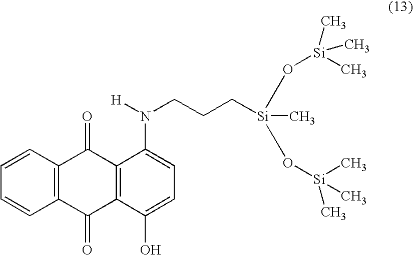
1,4-bis[3-[1,3,3,3-tetramethyl-1-[(trimethylsilyl)oxy]disiloxanyl]-propylamino]anthraquinone [compound (14)]: 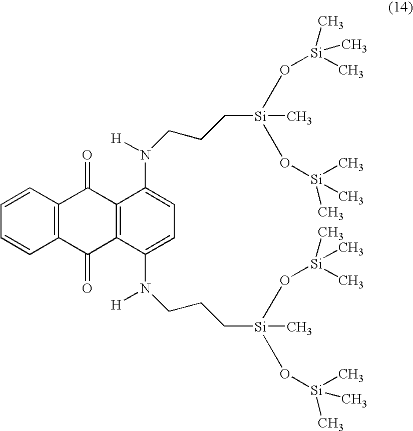
   VIII Nitrobenzene Dyes

Included among the nitrobenzene direct dyes that may be used according to the invention, particular mention may be made of those described in patent application WO 97/34904, of formula (15) or (16): 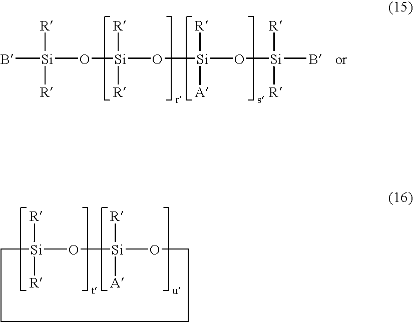
in which:
R′, which may be identical or different, are selected from the group consisting of linear or branched C₁-C₁₀ alkyl, phenyl and 3,3,3-trifluoropropyl radicals, at least 80% in numerical terms of the radicals R′ being methyl,
B′, which may be identical or different, are selected from the group consisting of the radicals R′ above and the radical A defined below,
r′ is an integer between 0 and 50 inclusive and s′ is an integer between 0 and 20 inclusive, with the condition that if s′ is zero then at least one of the two symbols B denotes A,
u′ is an integer between 1 and 6 inclusive, and t′ is an integer between 0 and 10 inclusive, it being understood that t′+u′ is greater than or equal to 3,
and the symbol A′ denotes a monovalent radical directly linked to a silicon atom, and which corresponds to formula (17) below: 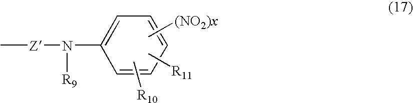
Z′ is a divalent radical: 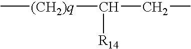
   or hydrogen,
x is 1 or 2,
q represents an integer between 0 and 10 inclusive,
R₉ represents hydrogen or a C₁-C₄ alkyl radical,
R₁₀ represents hydrogen or a C₁-C₄ alkyl radical, or the divalent radical Z′ defined above,
R₁₁ represents hydrogen or a radical NR₁₂R₁₃ in which R₁₂ and R₁₃ represent hydrogen or a C₁-C₄ alkyl radical, a C₂-C₄ monohydroxyalkyl or dihydroxyalkyl radical or a divalent radical Z, it being understood that at least one radical Z, [lacuna]
R₁₄ represents hydrogen, an OH or halogen radical, a C₁-C₄ alkyl radical or a C₁-C₄ alkoxy radical.

According to the invention, the compounds of formula (15) that are more particularly preferred are the following: 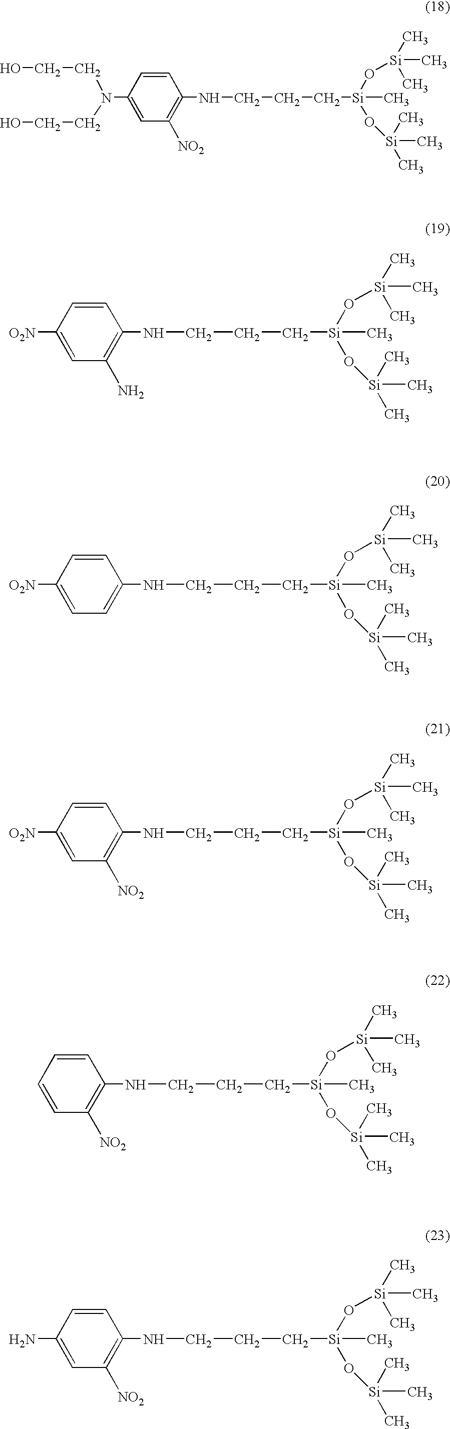
IX Compounds of the Aryl Vinylene Ketone Type

Included among the organic compounds of the aryl vinylene ketone type capable of screening out radiation preferably ranging from 380 to 500 nm according to the invention, particular mention may be made of those described in patent application FR 2 827 510, corresponding to one of the formulae (I) and (II) below: 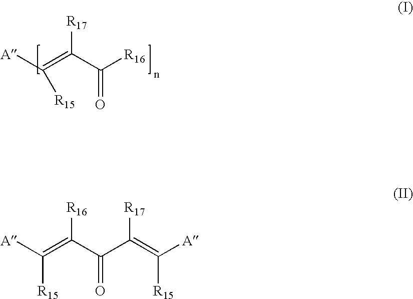
in which:
n=1 or 2,
A″, in formula (I) when n=1 or in formula (II), is an aryl radical selected from the group consisting of formulae (a) to (d) below, or in formula (I) when n=2, is a radical selected from the group consisting of formulae (e) to (h) below: 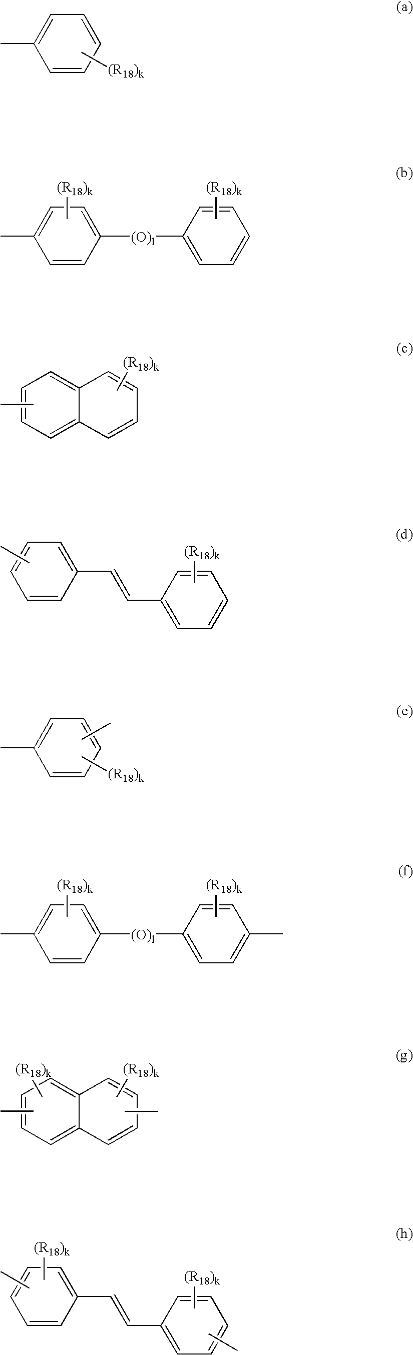
in which:
each of the symbols R₁₈ independently represents an OH group, a halogen atom, a linear or branched C₁₋₆ alkyl group optionally containing a silicon atom or a siloxane group, a linear or branched C₁₋₆ alkoxy group optionally containing a silicon atom or a siloxane group, a linear or branched C₁₋₅ alkoxycarbonyl group, or a linear or branched C₁₋₆ alkylsulfonamide group optionally containing a silicon atom, a siloxane group or an amino acid function,
k represents an integer between 0 and 3 inclusive,
1 represents 0 or 1,
R₁₅ represents hydrogen or an OH group,
R₁₆ represents hydrogen, a linear or branched C₁₋₆ alkyl group optionally containing a silicon atom or a siloxane group, a cyano group, a C₁₋₆ alkylsulfonyl group or a phenylsulfonyl group,
R₁₇ represents a linear or branched C₁₋₆ alkyl group optionally containing a silicon atom, a siloxane group or a phenyl group that can form a bicycle and optionally substituted with one or two radicals R₁₈ as defined above,
R₁₆ and R₁₇ may together form a monocyclic, bicyclic or tricyclic C₂₋₁₀ hydrocarbon-based residue, optionally interrupted with one or more nitrogen, sulfur and oxygen atoms and possibly containing another carbonyl, and optionally substituted with a linear or branched C₁-C₈ alkylsulfonamide group, optionally containing a silicon atom, a siloxane group or an amino acid function; on condition that when n=1, R₁₆ and R₁₇ do not form a camphor nucleus.

As examples of insoluble compounds of formula (I) in which n=1, which may be used according to the invention, particular mention may be made of the following families:
Styryl ketone (Kao JP 04 134 042) such as 1-(2,4,5-trimethoxyphenyl)-4,4-dimethylpent-1-en-3-one (λₘₐₓ 362 nm): 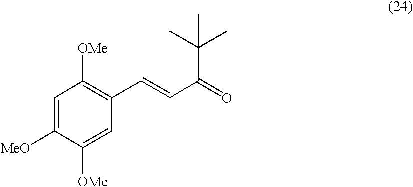
Benzylidene chromanone (Kao JP 04 134 043) such as 3-(3-ethoxy-4-butoxybenzylidene)-2,3,4a,8a-tetrahydrochromen-4-one (λₘₐₓ 370 nm): 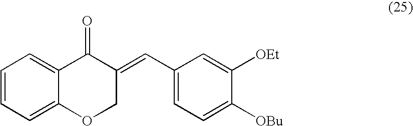
Benzylidene thiochromanone (Kao JP 04 134 043) such as 3-(4-methoxybenzylidene)-2,3,4a,8a-tetrahydrochromen-4-thione: 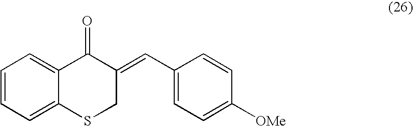
Benzylidene indanone (Kao JP 04 134 043) such as 2-(3-methoxy-4-butoxybenzylidene)indan-1-one: 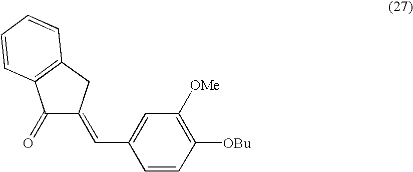
Benzylidene tetralone (Kao JP 04 134 043) such as 2-(3-methoxy-4-butoxybenzylidene)-3,4-dihydro-2H-naphthalen-1-one: 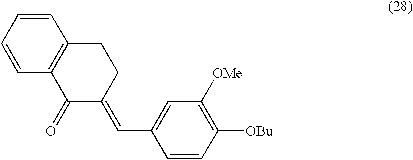
Benzylidene benzofuranone (Kao JP 04 134 041) such as 2-benzylidenebenzofuran-3-one (λₘₐₓ 395 nm): 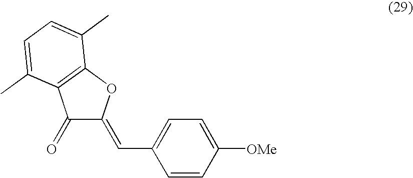
Benzylidene indanedione such as 2-(3,5-dimethoxy-4-hydroxybenzylidene)indan-1,3-dione: 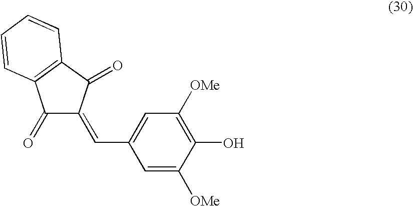
Benzylidene benzothiofuranone (Kao JP 04 134 043) such as 2-benzylidenebenzo[b]thiophen-3-one: (λₘₐₓ 428 nm): 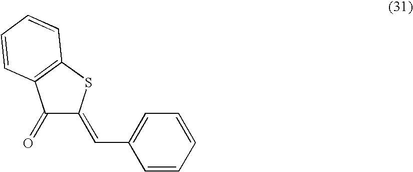
Benzylidene pyrazolone such as 4-(3-methoxy-4-butoxybenzylidene)-5-methyl-2-phenyl-2,4-dihydropyrazol-3-one: 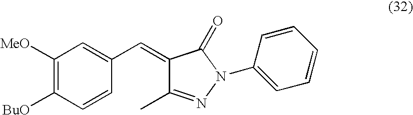
Benzylidene imidazolone such as 5-(3-methoxy-4-butoxybenzylidene)-2-phenyl-3,5-dihydroimidazol-4-one: 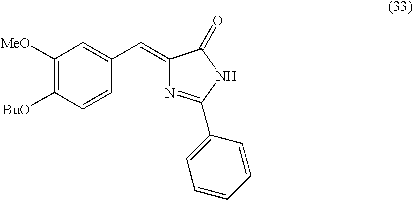
Chalcone such as 1-(2-hydroxy-4-methoxyphenyl)-3-(4′-methoxyphenyl)propenone: 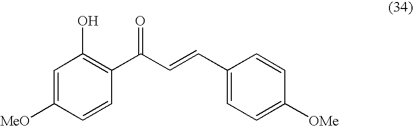
Benzylidene one (screening tautomeric form of the dibenzoylmethanes; L'Oréal FR 2 506 156) such as 3-hydroxy-1-(2-hydroxy-4-methoxyphenyl)-3-phenylpropenone:

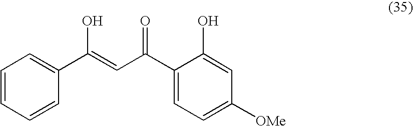

As examples of compounds of formula (I) in which n=2 that may be used according to the invention, particular mention may be made of the following families:
para-xylidene ketones (Kao JP 04 134 041), such as compound (36) below (λₘₐₓ 380 nm): 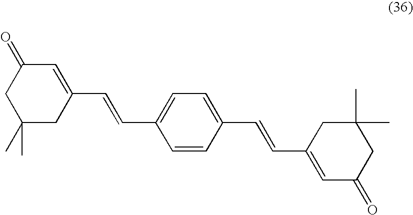
phenylenebis(methylidenenorcamphor) (Merck EP 0 693 471), such as 2,5-dimethoxyphenylene-1,4-bis(3-methylidenebicyclo[2.2.1]heptan-2-one): 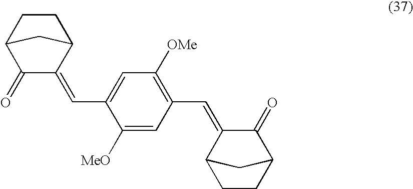
phenylenebis(methylidenecamphor) (L'Oréal FR 2 528 420), such as 2,5-dihexyloxyphenylene-1,4-bis(3-methylidene-1,7,7-trimethylbicyclo[2.2.1]heptan-2-one):

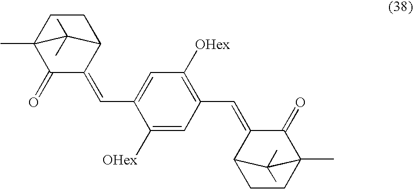

Included among compounds of formula (II), particular mention may be made of the following families:
bis(benzylidene)cycloalkanone (Shiseido JP 88 051 320), such as 2,5-bis(4-hexyloxybenzylidene)cyclopentanone:

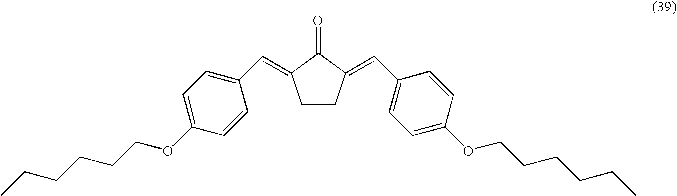

Particular mention may also be made of 2,5-bis[4-(3-trimethylsilanylpropyloxy)benzylidene]cyclopentanone (λₘₐₓ=395 nm): 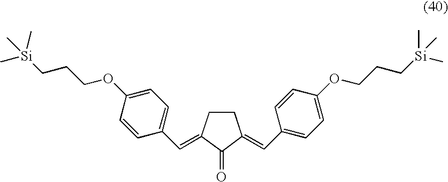
X Aminoarylvinyl-s-triazine Compounds

Included among the aminoarylvinyl-s-triazine compounds that may be used more particularly are those corresponding to formula (III) below: 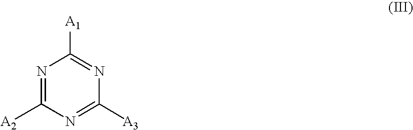
in which:
A₁, A₂ and A₃, which may be identical or different, are selected from the group consisting of the groups of formulae (IV) to (VII) below, it being understood that at least one group of formulae (IV) to (VII) is present: 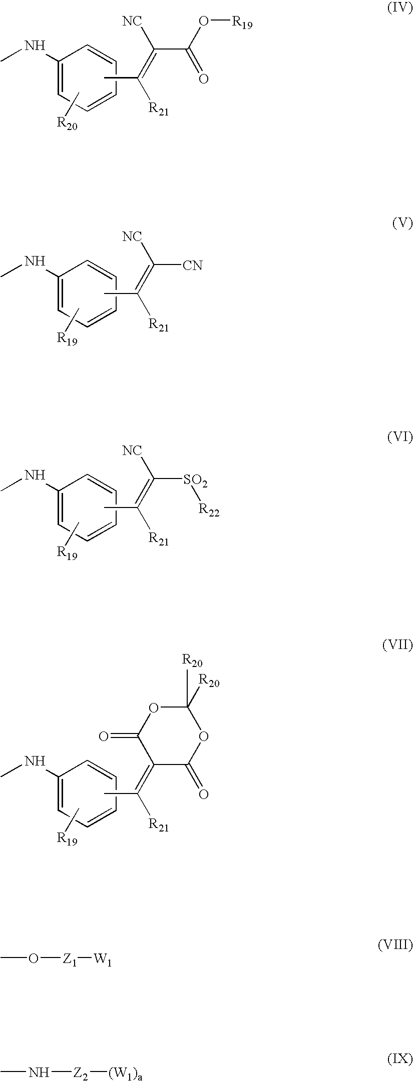
   in which:
R₂₀ represents hydrogen, a C₁-C₄ alkyl radical or a C₁-C₄ alkoxy radical,
R₁₉ represents a linear or branched C₁-C₂₀ alkyl radical, a linear or branched C₂-C₂₀ hydroxyalkyl radical or a linear or branched C₁-C₂₀ alkoxy radical,
R₂₁ represents hydrogen, a methyl radical or a phenyl radical optionally substituted with a C₁-C₄ alkyl radical or a C₁-C₄ alkoxy radical,
R₂₂ represents a linear or branched C₁-C₂₀ alkyl radical or a phenyl radical optionally substituted with a C₁-C₄ alkyl radical or a C₁-C₄ alkoxy radical,
Z₁ represents a divalent radical providing the bond between —O— and —W₁,
Z₂ represents a divalent radical when a=1 and a trivalent radical when a=2, providing the bond between —NH— and —(W₁)a,
Z₁ and Z₂ possibly being C₁-C₁₂ alkylene, optionally substituted with one or more hydroxyl groups and possibly containing one or more oxygen atoms or one or more amino groups and optionally containing a double bond,
W₁ represents:
(i) either a silicone radical comprising at least one unit of formula (X) below 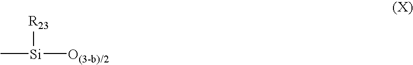
   in which R₂₃ denotes a saturated or unsaturated, linear or branched C₁-C₃₀ hydrocarbon-based group; a C₁-C₈ halohydrocarbon group; a phenyl radical; a 3,3,3-trifluoropropyl radical or a trimethylsilyloxy group, and b is equal to 1 or 2, (ii) or a radical of formula (XI) below:
   —SiR₂₄R₂₅R₂₆ (XI)
   in which R₂₄, R₂₅ and R₂₆, which may be identical or different, are selected from the group consisting of linear or branched C₁-C₈ alkyl and alkenyl radicals.

The groups A₁, A₂ and A₃ may also represent a UVB- and/or UVA-absorbing chromophore preferably selected from the group consisting of aryl groups substituted with hydroxyl groups, linear or branched C₁-C₂₀ alkyl groups and linear or branched C₁-C₂₀ alkoxy groups; aminobenzylidenecamphor groups; aminobenzotriazole groups; linear or branched aminobenzoate, aminobenzalmalonate, aminosalicylate, C₁-C₂₀ aminocinnamate or anthranilate ester groups.

According to one preferred form of the invention, W is a silicone radical corresponding to one of the three formulae (XII) to (XIV) below: 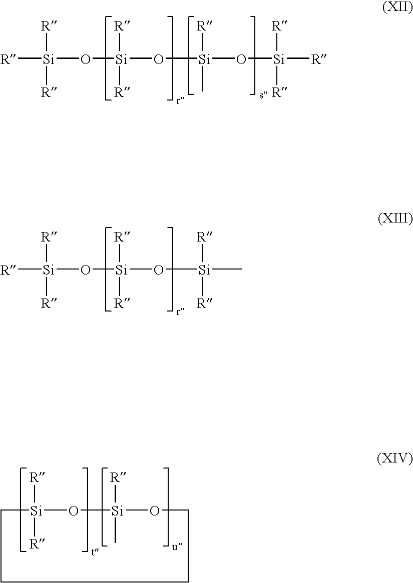
in which:
R″, which may be identical or different, are selected from the group consisting of linear or branched, saturated or unsaturated C₁-C₂₀ alkyl radicals, a phenyl radical and a 3,3,3-trifluoropropyl radical, at least 80% in numerical terms of the radicals R being methyl radicals,
r″ is an integer chosen between 0 and 50 inclusive,
s″ is an integer chosen between 0 and 20 inclusive,
u″ is an integer between 1 and 6 inclusive,
t″ is an integer between 0 and 10 inclusive,
t″+u″ is greater than or equal to 3.

Included among the compounds of formula (III) above that are preferably used are those for which the radical W corresponds to formula (XII) or formula (XIII), i.e. those for which the silicone radical is a linear diorganosiloxane radical.

Included among the linear diorganosiloxane radicals falling within the context of the present invention, the ones more particularly preferred are the random derivatives or derivatives with well-defined blocks having at least one and even more preferably all of the following characteristics:
R″ is alkyl and even more preferably is methyl,
r″ is between 0 and 3 inclusive; s is between 0 and 3 inclusive.

The compounds of formula (III) that are more particularly preferred are selected from the group consisting of:
2-(ethyl 4′-ylamino-α-cyanocinnamate)-4-(4-methoxyphenyl)-6-{[1,3,3,3-tetramethyl-1-[(trimethylsilyl)oxy]disiloxanyl]propyl-3-ylamino}-s-triazine,
2-(ethyl 4′-ylamino-α-cyanocinnamate)-4-(butyl 4′-ylaminobenzoate)-6-{[1,3,3,3-tetramethyl-1-[(trimethylsilyl)oxy]disiloxanyl]propyl-3-ylamino}-s-triazine,
2-(4′-ylamino-2-methanesulfonylacrylonitrile)-4-(butyl 4′-ylaminobenzoate)-6-{[1,3,3,3-tetramethyl-1-[(trimethylsilyl)oxy]disiloxanyl]propyl-3-ylamino}-s-triazine,
2-(ethyl 4′-ylamino-α-cyanocinnamate)-4-(butyl 4′-ylaminobenzoate)-6-(trimethylsilanylmethylylamino)-s-triazine,
2,4-bis(butyl 4′-diylaminobenzoate)-6-(2-ethylhexyl 4′-ylamino-α-cyanocinnamate)-s-triazine,
2,4,6-tris(isobutyl 4′-ylamino-α-cyanocinnamate)-s-triazine,
2,4,6-tris(2-ethylhexyl 4′-ylamino-α-cyanocinnamate)-s-triazine,
2,4-bis(2-ethylhexyl 4′-ylamino-α-cyanocinnamate)-6-{[1,3,3,3-tetramethyl-1-[(trimethylsilyl)oxy]disiloxanyl]propyl-3-ylamino}-s-triazine.

Among the compounds of formula (III), some are known per se in the chemical literature of s-triazine compounds. These are:
the compounds of formula (III) in which the 3 groups A₁, A₂ and A₃ correspond to the same formula (IV)
the compounds of formula (III) in which A₂ and A₃ denote a group of formula (IV) and A₁ denotes a group of formula (VIII) or (IX).

Apart from these compounds, the molecules of formula (III) are novel and constitute a subject of the invention. Included among the novel compounds of formula (III) that may be particularly mentioned are:
2,4-bis(butyl 4′-diylaminobenzoate)-6-(2-ethylhexyl 4′-ylamino-α-cyanocinnamate)-s-triazine;
2-(4′-ylamino-2-methanesulfonylacrylonitrile)-4-(butyl 4′-ylaminobenzoate)-6-{[1,3,3,3-tetramethyl-1-[(trimethylsilyl)oxy]disiloxanyl]propyl-3-ylamino}-s-triazine.

The compounds of formula (III) may be obtained according to the reaction scheme below: 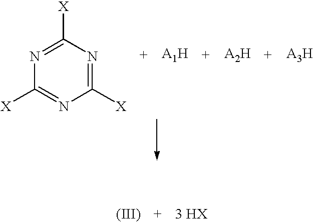
in which A₁, A₂ and A₃ correspond to the above definitions and X represents a halogen, in particular chlorine or bromine.

The grafting of the various radicals A₁, A₂ and A₃ above onto the s-triazine may take place independently of each other and in any order.

Preferably, the radical —O—Z₁—W₁ or the radical —NH—Z₂—(W₁)ₐ corresponding to aminosilicone or aminosilane chains is grafted onto the s-triazine first, followed by the radicals A₂ and A₃.

In the particular case of siloxane derivatives, this group may be introduced onto s-triazine via the aminosiloxane derivative as described in the preceding scheme, or alternatively it is introduced via unsaturated amino derivatives of formulae (XIV) and (XV) below: 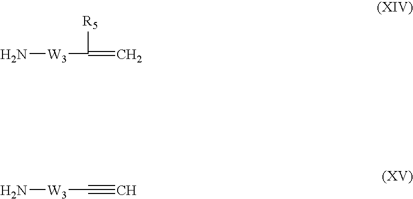
followed by a hydrosilylation reaction of the introduced unsaturation with a derivative of formula (XVI): 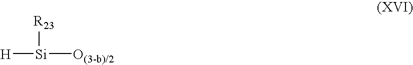
The preparation of the aminosiloxanes is described, for example, in GB 2 185 984. As aminosiloxanes that are particularly suitable for preparing the compounds according to the invention, mention may be made of aminopropylheptamethyltrisiloxane, aminoisobutylheptamethyltrisiloxane or trimethylsilylamodimethicones, such as: the product sold under the trade name “X2-8260” by the company Dow Corning, with an amine number of 2.8 meq/gram; the product sold under the trade name “SLM 55051/3” by the company Wacker, with an amine number of 0.47 meq/gram; C12 dimethylalkyl PDMSs, such as the product sold under the trade name “SLM 23046/1” by the company Wacker, with an amine number of 1.2 meq/gram; α,ω-trimethyl polymethylalkyl (fatty) aryl-alkylsiloxanes, such as the product sold under the trade name “SLM 23056/2” by the company Wacker, with an amine number of 1.3 meq/gram; PDMSs in which the radical NH2 is in the α and ω positions on an alkyl site, such as the products sold under the trade names “Tegomer A-SI 2120”, with an amine number of 1.95 meq/gram, and “Tegomer A-SI 2320”, with an amine number of 0.86 meq/gram, by the company Goldschmidt.

The preparation of the cyclic aminosiloxanes is described, for example, in the article by A. Kopylov, Zh. Obshch. Khim., 54(2), 367-71 (1984).

The preparation of the aminosilanes is described, for example, in EP 321 174 or in the article by J. P. Picard, Can. J. Chem., 78(11), 1363-1379 (2000).

As aminosilane derivatives that are particularly suitable for preparing the compounds of the present invention, mention may be made of amino-propyltrimethylsilane, aminomethyltrimethylsilane and 1,1-bis(trimethylsilyl)methylamine.

The above reactions may optionally be performed in the presence of a solvent, for instance toluene or xylene, or alternatively an acetone/water mixture.

The above reactions may also optionally be performed in the presence of a base such as sodium hydroxide, carbonates or an amine.

The compounds A₁H to A₃H corresponding to the radicals A₁ to A₃ may be prepared according to known methods.

The preparation of the amino cyanoacrylate derivatives is described, for example, in the article J. Soc. Dyers Colour. (1977), 93, pp. 126-133. As amino cyanoacrylate derivatives that are particularly suitable for preparing the compounds of the present invention, mention may be made of 2-ethylhexyl α-cyano-4-aminocinnamate.

The agents for screening out light radiation with a wavelength ranging from 370 to 500 nm according to the invention are preferably present in the compositions in amounts that reduce the amount of light radiation with a wavelength ranging from 370 to 500 nm that reach the skin. The preferred % reduction is thus preferably greater than 0, and includes all values thereabove such as 5%, 10%, 20%, 30%, 40%, 50%, 60%, 70%, 80%, 90%, 95% and 100%. One of ordinary skill is able to provide such amounts without undue experimentation in view of this disclosure. While the amount of the agent(s) is not limited, examples of concentrations ranging from 0.1% to 15% by weight and more particularly from 0.1% to 10% by weight relative to the total weight of the composition can be given as a guide.

The compositions according to the invention are preferably suitable for external topical application. They may be in any form, for example any galenical form applied to the surface of the skin that is usually proposed in, e.g., cosmetics and dermatology. They may especially comprise at least one fatty phase and may be in the form of an oily solution, an oil-in-water, water-in-oil or multiple emulsion, a silicone emulsion, a microemulsion or nanoemulsion, an oily gel or a liquid, pasty or solid anhydrous product.

The compositions according to the invention may be cosmetic products for protecting the natural complexion of the skin.

The compositions according to the invention may also be cosmetic products for natural antioxidant protection of the skin.

The compositions according to the invention may be dermatological products for treating skin disorders caused by sunlight (photodermatoses), for instance solar urticaria, actinic dermatitis and pigmentation marks.

The compositions in accordance with the invention may also comprise at least one UVA-active and/or UVB-active organic photoprotective agent and/or at least one UVA-active and/or UVB-active mineral photoprotective agent (absorbers), which is (are) water-soluble or liposoluble or even insoluble in the cosmetic solvents commonly used.

The organic photoprotective agents may especially be selected from the group consisting of anthranilates; cinnamic derivatives; dibenzoylmethane derivatives; salicylic derivatives; camphor derivatives; triazine derivatives such as those described in patent applications U.S. Pat. No. 4,367,390, EP 863 145, EP 517 104, EP 570 838, EP 796 851, EP 775 698, EP 878 469, EP 933 376, EP 507 691, EP 507 692, EP 790 243 and EP 944 624; benzophenone derivatives; β,β′-diphenylacrylate derivatives; benzotriazole derivatives; benzalmalonate derivatives; benzimidazole derivatives; imidazolines; bis-benzazolyl derivatives as described in patents EP 669 323 and U.S. Pat. No. 2,463,264; p-aminobenzoic acid (PABA) derivatives; methylenebis(hydroxyphenyl)benzotriazole derivatives as described in patent applications U.S. Pat. Nos. 5,237,071, 5,166,355, GB 2 303 549, DE 197 26 184 and EP 893 119; benzoxazole derivatives as described in patent applications EP 0 832 642, EP 1 027 883, EP 1 300 137 and DE 101 62 844; screening polymers and screening silicones such as those described especially in patent application WO 93/04665; dimers derived from α-alkylstyrene such as those described in patent application DE 198 55 649; 4,4-diarylbutadienes such as those described in patent applications EP 0 967 200, DE 197 55 649, EP 1 133 980 and EP 1 133 981, and mixtures thereof.

Included among examples of UV-A-active and/or UV-B-active photoprotective agents, particular mention may be made of the following, denoted hereinbelow under their INCI name:

Para-Aminobenzoic Acid Derivatives:

PABA,
Ethyl PABA,
Ethyl dihydroxypropyl PABA,
Ethylhexyl dimethyl PABA sold in particular under the name “Escalol 507” by ISP,
Glyceryl PABA,
PEG-25 PABA sold under the name “Uvinul P25” by BASF.
   Salicylic Derivatives:
Homosalate sold under the name “Eusolex HMS” by Rona/EM Industries,
Ethylhexyl salicylate sold under the name “Neo Heliopan OS” by Haarmann and Reimer,
Dipropylene glycol salicylate sold under the name “Dipsal” by Scher,
TEA salicylate sold under the name “Neo Heliopan TS” by Haarmann and Reimer.
   Dibenzoylmethane Derivatives:
Butyl methoxydibenzoylmethane sold in particular under the trade name “Parsol 1789” by Hoffinann LaRoche,
Isopropyldibenzoylmethane.
   Cinnamic Derivatives:
Ethylhexyl methoxycinnamate sold in particular under the trade name “Parsol MCX” by Hoffmann LaRoche,
Isopropyl methoxycinnamate,
Isoamyl methoxycinnamate sold under the trade name “Neo Heliopan E 1000” by Haarmann and Reimer,
Cinoxate,
DEA methoxycinnamate,
Diisopropyl methylcinnamate,
Glyceryl ethylhexanoate dimethoxycinnamate.
   β,β′-Diphenyl Acrylate Derivatives:
Octocrylene sold in particular under the trade name “Uvinul N539” by BASF,
Etocrylene sold in particular under the trade name “Uvinul N35” by BASF.
   Benzophenone Derivatives:
Benzophenone-1 sold under the trade name “Uvinul 400” by BASF,
Benzophenone-2 sold under the trade name “Uvinul D50” by BASF,
Benzophenone-3 or Oxybenzone sold under the trade name “Uvinul M40” by BASF,
Benzophenone-4 sold under the trade name “Uvinul MS40” by BASF,
Benzophenone-5,
Benzophenone-6 sold under the trade name “Helisorb 11” by Norquay,
Benzophenone-8 sold under the trade name “Spectra-Sorb UV-24” by American Cyanamid,
Benzophenone-9 sold under the trade name “Uvinul DS-49” by BASF,
Benzophenone-12,
n-hexyl 2-(4-diethylamino-2-hydroxybenzoyl)benzoate.
   Benzylidenecamphor Derivatives:
3-Benzylidenecamphor manufactured under the name “Mexoryl SD” by Chimex,
4-Methylbenzylidenecamphor sold under the name “Eusolex 6300” by Merck,
Benzylidenecamphorsulfonic acid manufactured under the name “Mexoryl SL” by Chimex,
Camphor benzalkonium methosulfate manufactured under the name “Mexoryl SO” by Chimex,
Terephthalylidenedicamphorsulfonic acid manufactured under the name “Mexoryl SX” by Chimex,
Polyacrylamidomethylbenzylidenecamphor manufactured under the name “Mexoryl SW” by Chimex.
   Benzimidazole Derivatives:
Phenylbenzimidazolesulfonic acid sold in particular under the trade name “Eusolex 232” by Merck,
Disodium phenyldibenzimidazoletetrasulfonate sold under the trade name “Neo Heliopan AP” by Haarmann and Reimer.
   Triazine Derivatives:
Anisotriazine sold under the trade name “Tinosorb S” by Ciba Specialty Chemicals,
Ethylhexyltriazone sold in particular under the trade name “Uvinul T150” by BASF,
Diethylhexylbutamidotriazone sold under the trade name “Uvasorb HEB” by Sigma 3V,
2,4,6-Tris(diisobutyl 4′-aminobenzalmalonate) s-triazine.
   Benzotriazole Derivatives:
Drometrizole trisiloxane sold under the name “Silatrizole” by Rhodia Chimie,
Methylenebis(benzotriazolyl)tetramethylbutylphenol sold in solid form under the trade name “MIXXIM BB/100” by Fairmount Chemical, or in micronized form as an aqueous dispersion under the trade name “Tinosorb M” by Ciba Specialty Chemicals.
   Anthranilic Derivatives:
Menthyl anthranilate sold under the trade name “Neo Heliopan MA” by Haarmann and Reimer.
   Imidazoline Derivatives:
Ethylhexyldimethoxybenzylidenedioxoimidazoline propionate.
   Benzalmalonate Derivatives:
Polyorganosiloxanes, containing benzalmalonate functions, such as polysilicone-15, sold under the trade name “Parsol SLX” by Hoffmann LaRoche,
   4,4-Diarylbutadiene Derivatives:

1,1-Dicarboxy(2,2′-dimethylpropyl)-4,4-diphenylbutadiene, and mixtures thereof.

The organic photoprotective agents that are more particularly preferred are selected from the group consisting of the following compounds:
Ethylhexyl salicylate,
Butyl methoxydibenzoylmethane,
Ethylhexyl methoxycinnamate,
Octocrylene,
Phenylbenzimidazolesulfonic acid,
Terephthalylidenedicamphorsulfonic acid,
Benzophenone-3,
Benzophenone-4,
Benzophenone-5,
n-Hexyl 2-(4-diethylamino-2-hydroxybenzoyl)benzoate,
4-Methylbenzylidenecamphor,
Disodium phenyldibenzimidazoletetrasulphonate,
Anisotriazine,
Ethylhexyltriazone,
Diethylhexylbutamidotriazone,
2,4,6-Tris(diisobutyl 4′-aminobenzalmalonate)-s-triazine,
Methylenebis(benzotriazolyl)tetramethylbutylphenol,
Drometrizole trisiloxane,
Polysilicone-15,
1,1-Dicarboxy(2,2′-dimethylpropyl)-4,4-diphenylbutadiene,
2,4-Bis[5-1(dimethylpropyl)benzoxazol-2-yl(4-phenyl)imino]-6-(2-ethylhexyl)imino-1,3,5-triazine,
   and mixtures thereof.

The mineral photoprotective agents are preferably selected from the group consisting of pigments or even nanopigments (mean size of the primary particles: generally between 5 nm and 100 nm and preferably between 10 nm and 50 nm) of coated or uncoated metal oxides such as, for example, nanopigments of titanium oxide (amorphous or crystallized in rutile and/or anatase form), of iron oxide, of zinc oxide, of zirconium oxide or of cerium oxide, which are all UV photoprotective agents that are well known per se. Standard coating agents are, moreover, alumina and/or aluminium stearate. Such coated or uncoated metal oxide nanopigments are described in particular in patent applications EP 518 772 and EP 518 773.

The treated nanopigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminium salts of fatty acids, metal (titanium or aluminium) alkoxides, poly-ethylene, silicones, proteins (collagen or elastin), alkanolamines, silicon oxides, metal oxides, sodium hexametaphosphate, alumina or glycerol.

The treated nanopigments may preferably be titanium oxides treated with:
silica and alumina, such as the products “Microtitanium Dioxide MT 500 SA” and “Microtitanium dioxide MT 100 SA” from the company Tayca, and the products “Tioveil Fin”, “Tioveil OP”, “Tioveil MOTG” and “Tioveil IPM” from the company Tioxide,
alumina and aluminium stearate, such as the product “Microtitanium Dioxide MT 100 T” from the company Tayca,
alumina and aluminium laurate, such as the product “Microtitanium Dioxide MT 100 S” from the company Tayca,
iron oxides and iron stearate, such as the product “Microtitanium Dioxide MT 100 F” from the company Tayca,
silica, alumina and silicone, such as the products “Microtitanium Dioxide MT 100 SAS”, “Microtitanium Dioxide MT 600 SAS” and “Microtitanium Dioxide MT 500 SAS” from the company Tayca,
sodium hexametaphosphate, such as the product “Microtitanium Dioxide MT 150 W” from the company Tayca,
octyltrimethoxysilane, such as the product “T-805” from the company Degussa,
alumina and stearic acid, such as the product “UVT-M160” from the company Kemira,
alumina and glycerol, such as the product “UVT-M212” from the company Kemira,
alumina and silicone, such as the product “UVT-M262” from the company Kemira.

The untreated titanium oxides may be, for example, those sold by the company Tayca under the trade name “Microtitanium Dioxide MT 500 B” or “Microtitanium Dioxide MT 600 B”.

The untreated zinc oxides may be, for example, those sold by the company Sumitomo under the name “Ultra Fine Zinc Oxide Powder”, by the company Presperse under the name “Finex 25”, by the company Ikeda under the name “MZO-25” or by the company Sunsmart under the name “Z-Cote”. The treated zinc oxides may be, for example, those sold by the company Sunsmart under the name “Z-Cote HP 1”.

The nanopigments may be introduced into the compositions according to the invention in unmodified form or in the form of pigmentary paste, i.e. as a mixture with a dispersant, as described, for example, in document GB-A-2 206 339.

The photoprotective agents are generally present in the compositions according to the invention in proportions ranging from 0.01% to 20% by weight relative to the total weight of the composition and preferably ranging from 0.1% to 10% by weight relative to the total weight of the composition.

The compositions according to the invention may also contain agents for artificially tanning and/or browning the skin (self-tanning agents) and more particularly dihydroxyacetone (DHA). They are preferably present in amounts ranging from 0.1% to 10% by weight relative to the total weight of the composition.

The compositions in accordance with the present invention may also comprise standard cosmetic adjuvants chosen especially from fatty substances, organic solvents, ionic or nonionic, hydrophilic or lipophilic thickeners, softeners, humectants, opacifiers, stabilizers, emollients, silicones, antifoams, fragrances, preserving agents, anionic, cationic, nonionic, zwitterionic or amphoteric surfactants, active agents, fillers, polymers, propellants, acidifying or basifying agents or any other ingredient usually used in cosmetics and/or dermatology.

The fatty substances may consist of an oil or a wax or mixtures thereof. The term “oil” means a compound that is liquid at room temperature. The term “wax” means a compound that is solid or substantially solid at room temperature and whose melting point is generally greater than 35° C.

Oils that may be mentioned include mineral oils (paraffin); plant oils (sweet almond oil, macadamia oil, grapeseed oil or jojoba oil); synthetic oils, for instance perhydrosqualene, fatty alcohols, fatty acids or fatty esters (for instance the C₁₂-C₁₅ alkyl benzoate sold under the trade name “Finsolv TN” by the company Witco, octyl palmitate, isopropyl lanolate and triglycerides, including capric/caprylic acid triglycerides), oxyethylenated or oxypropylenated fatty esters and ethers; silicone oils (cyclomethicone and polydimethylsiloxanes, or PDMS) or fluoro oils, and polyalkylenes.

Waxy compounds that may be mentioned include paraffin, carnauba wax, beeswax and hydrogenated castor oil.

Among the organic solvents that may be mentioned are lower alcohols and polyols. These polyols may be selected from the group consisting of glycols and glycol ethers, for instance ethylene glycol, propylene glycol, butylene glycol, dipropylene glycol or diethylene glycol.

Hydrophilic thickeners that may be mentioned include carboxyvinyl polymers such as the Carbopol products (carbomers) and the Pemulen products (acrylate C10-C30-alkylacrylate copolymer); polyacrylamides, for instance the crosslinked copolymers sold under the names Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) by the company SEPPIC; 2-acrylamido-2-methylpropanesulfonic acid polymers and copolymers, which are optionally crosslinked and/or neutralized, for instance the poly(2-acrylamido-2-methylpropanesulfonic acid) sold by the company Hoechst under the trade name “Hostacerin AMPS” (CTFA name: ammonium polyacryldimethyltauramide); cellulose-based derivatives such as hydroxyethylcellulose; polysaccharides and especially gums such as xanthan gum; and mixtures thereof.

Lipophilic thickeners that may be mentioned include modified clays such as hectorite and its derivatives, for instance the products sold under the name bentone.

Among the active agents that may be mentioned are:
antipollution agents and/or free-radical scavengers;
depigmenting agents and/or propigmenting agents;
antiglycation agents;
NO-synthase inhibitors;
agents for stimulating the synthesis of dermal or epidermal macromolecules and/or for preventing their degradation;
agents for stimulating fibroblast proliferation;
agents for stimulating keratinocyte proliferation;
muscle relaxants;
tensioning agents;
desquamating agents;
moisturizers;
anti-inflammatory agents;
agents acting on the energy metabolism of cells;
insect repellants;
substance P or CGRP antagonists.

Needless to say, a person skilled in the art will take care to select the optional additional compound(s) mentioned above and/or the amounts thereof such that the advantageous properties intrinsically associated with the compounds in accordance with the invention are not, or are not substantially, adversely affected by the envisaged addition(s).

The compositions according to the invention may be prepared according to techniques that are well known to those skilled in the art in view of this disclosure, in particular those intended for the preparation of emulsions of oil-in-water or water-in-oil type. They may be in particular in the form of a simple or complex emulsion (O/W, W/O, O/W/O or W/O/W emulsion) such as a cream or a milk, in the form of a gel or a cream-gel, or in the form of a lotion, a powder or a solid tube, and may optionally be packaged as an aerosol and may be in the form of a mousse or a spray.

The compositions according to the invention are preferably in the form of an oil-in-water or water-in-oil emulsion.

The emulsions generally contain at least one emulsifier selected from the group consisting of amphoteric, anionic, cationic and nonionic emulsifiers, which are used alone or as a mixture. The emulsifiers are appropriately chosen according to the emulsion to be obtained (W/O or O/W).

As emulsifying surfactants that may be used for the preparation of the W/O emulsions, examples that may be mentioned include sorbitan, glycerol or sugar alkyl esters or ethers; silicone surfactants, for instance dimethicone copolyols, such as the mixture of cyclomethicone and of dimethicone copolyol, sold under the name “DC 5225 C” by the company Dow Corning, and alkyldimethicone copolyols such as laurylmethicone copolyol sold under the name “Dow Corning 5200 Formulation Aid” by the company Dow Corning; cetyl-dimethicone copolyol, such as the product sold under the name Abil EM 90R by the company Goldschmidt, and the mixture of cetyldimethicone copolyol, of poly-glyceryl isostearate (4 mol) and of hexyl laurate, sold under the name Abil WE O9 by the company Goldschmidt. One or more co-emulsifiers may also be added thereto, which may be chosen advantageously from the group comprising polyol alkyl esters. Polyol alkyl esters that may especially be mentioned include glycerol and/or sorbitan esters, for example polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

For the O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alkyl ethers; sugar esters, for instance sucrose stearate; fatty alkyl ethers of sugars, especially polyalkylglucosides (APG) such as decylglucoside and laurylglucoside sold, for example, by the company Henkel under the respective names Plantaren 2000 and Plantaren 1200, cetostearylglucoside optionally as a mixture with cetostearyl alcohol, sold, for example, under the name Montanov 68 by the company SEPPIC, under the name Tegocare CG90 by the company Goldschmidt and under the name Emulgade KE3302 by the company Henkel, and also arachidylglucoside, for example in the form of a mixture of arachidyl alcohol, behenyl alcohol and arachidylglucoside, sold under the name Montanov 202 by the company SEPPIC. According to one particular embodiment of the invention, the mixture of the alkylpolyglucoside as defined above with the corresponding fatty alcohol may be in the form of a self-emulsifying composition as described, for example, in document WO-A-92/06778.

When it is an emulsion, the aqueous phase of this emulsion may comprise a nonionic vesicular dispersion prepared according to known processes (Bangham, Standish and Watkins, J. Mol. Biol. 13, 238 (1965), FR 2 315 991 and FR 2 416 008).

Concrete, but in no way limiting, examples illustrating the invention will now be given.

EXAMPLE 1
Synthesis of 2,5-bis[4-(3-trimethylsilanyl-propyloxy)benzylidene]cyclopentanone 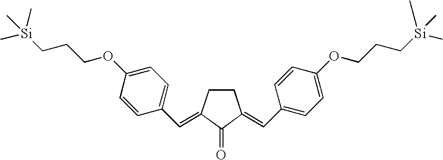
First Step: Preparation of 4-(3-trimethylsilanylpropyloxy)benzaldehyde:

3-Chloropropyltrimethylsilane (33.14 g, 0.22 mol) is added dropwise over 10 minutes to a mixture of 4-hydroxybenzaldehyde (24.4 g, 0.2 mol) and potassium carbonate (30.4 g, 0.22 mol) in 150 ml of dry DMF brought to 120° C. under nitrogen. The mixture is left for 2 hours 30 minutes at 120-130° C. The reaction mixture is cooled and poured into ice-water. The aqueous phase is extracted three times with dichloromethane. The organic phases are dried over sodium sulfate and concentrated under vacuum. After distillation under vacuum (0.2 mmHg), 40.5 g (yield: 86%) of 4-(3-trimethylsilanylpropyloxy)benzaldehyde are obtained in the form of a colourless oil that distils at 110-114° C., which is used without further purification in the following step.

Second Step: Preparation of the Derivative of Example 1:

4.2 g of sodium hydroxide (0.105 mol) dissolved in 22 ml of water are added dropwise over 15 minutes to a mixture of the preceding product (26.2 g, 0.105 mol) and of cyclopentanone (4.2 g, 0.05 mol) in 200 ml of methanol at 35° C., under nitrogen, with stirring. The mixture is refluxed for 6 hours. It is cooled and the yellow precipitate obtained is filtered off and washed thoroughly with water. After drying, 25 g (yield: 96%) of the derivative of Example 1 are obtained in the form of a yellow solid.
Melting point: 205-207° C.
UV (CHCl₃) λₘₐₓ=395 mn,εₘₐₓ=49 100, E1% =940.
Elemental analysis for C₃₁H₄₄O₃Si₂
calculated: C 71.49 H 8.51 Si 10.78
found: C 71.15 H 8.78 Si 10.47.

### EXAMPLE 2

Preparation of 2-(ethyl 4′-ylamino-α-cyanocinnamate)-4-(4-methoxyphenyl)-6-{[1,3,3,3-tetramethyl-1-[(trimethylsilyl)oxy]disiloxanyl]propyl-3-ylamino}-s-triazine:

First Step: Preparation of Ethyl 3-{4-[4-chloro-6-(methoxyphenyl)[1,3,5]triazin-2-ylamino]phenyl}-2-cyanoacrylate:

A solution of ethyl 3-(4-aminophenyl)-2-cyanoacrylate (5.4 g, 0.025 mol) dissolved in 50 ml of acetone and 25 ml of aqueous sodium bicarbonate solution (2.1 g, 0.025 mol) are alternately added dropwise, over 1 hour, to a solution of 2,4-dichloro-6-(4-methoxyphenyl)[1,3,5]triazine (6.4 g, 0.025 mol) in 60 ml of acetone at 50° C. Stirring is continued for 1 hour. After filtering off the precipitate by suction, washing with water and drying, the ethyl 3-{4-[4-chloro-6-(methoxyphenyl)[1,3,5]triazin-2-ylamino]phenyl}-2-cyanoacrylate (7.5 g, yield=94%) is obtained, this product having the following characteristics;
Yellow solid
UV (DMSO/CH₂Cl₂) λₘₐₓ=375 nm,εₘₐₓ=36 850
   Second Step: Preparation of the Compound of Example 2:

The preceding derivative (4.3 g, 0.01 mol) is suspended in 50 ml of toluene. 1-Amino-3-[1,3,3,3-tetramethyl-1-[(trimethylsilyl)oxy]disiloxanyl]propane (5.6 g, 0.02 mol) is added thereto and the mixture is heated at 90° C. with stirring for 2 hours, while sparging with nitrogen. The resulting mixture is evaporated to dryness and the oil obtained is taken up in heptane. The precipitate obtained is purified by chromatography on a column of silica (eluent: CH₂Cl₂). 3.2 g (yield:

47%) of the derivative of Example 2 are obtained in the form of a bright yellow solid:
UV (ethanol) λₘₐₓ=390 nm,εₘₐₓ=44 640
Elemental analysis for C₃₂H₄₆N₆O₅Si₃
calculated: C 56.61 H 6.83 N 12.38 Si 12.41
found: C 56.32 H 6.89 N 12.40 Si 12.60

### EXAMPLE 3

Preparation of 2-(ethyl 4′-ylamino-α-cyanocinnamate)-4-(butyl 4′-ylaminobenzoate)-6-{[1,3,3,3-tetramethyl-1-[(trimethylsilyl)oxy]disiloxanyl]propyl-3-ylamino}-s-triazine:

First Step: Preparation of Ethyl 3-{4-[4-chloro-6-(4-pentanoylphenylamino)-[1,3,5]triazin-2-ylamino]phenyl}-2-cyanoacrylate:

A solution of ethyl 3-(4-aminophenyl)-2-cyanoacrylate (12.5 g, 0.058 mol) dissolved in 120 ml of acetone and 25 ml of aqueous sodium bicarbonate solution (4.9 g, 0.058 mol) are alternately added dropwise over 1 hour at 50° C. to a solution of 2-(butyl 4′-ylaminobenzoate)-4,6-dichloro-s-triazine (19.7 g, 0.058 mol) in 50 ml of DMF brought to 50° C. Stirring is continued for 2 hours at 80° C. The acetone is removed and the reaction mixture is diluted with water. The solid obtained is filtered off, washed with water and dried. The ethyl 3-{4-[4-chloro-6-(4-pentanoylphenylamino)-[1,3,5]triazin-2-ylamino]phenyl}-2-cyanoacrylate (12.5 g, yield=41%) is obtained, this product having the following characteristics:
Yellow solid
UV (DMSO/CH₂Cl₂) λₘₐₓ=376 nm,εₘₐₓ=26 870
λₘₐₓ=297 nm,εₘₐₓ=28 180
   Second Step:

The preceding derivative (10.4 g, 0.02 mol) is suspended in 100 ml of toluene. 1-Amino-3-[1,3,3,3-tetramethyl-1-[(trimethylsilyl)oxy]disiloxanyl]propane (11.2 g, 0.04 mol) is added thereto and the mixture is heated at 90° C. with stirring for 2 hours, while sparging with nitrogen. The resulting mixture is evaporated to dryness and the oil obtained is taken up in heptane. The paste obtained is purified by chromatography twice on a column of silica (eluent: 70/30 heptane/EtOAc). 2 g (yield: 12%) of the product of EXAMPLE 3 are obtained in the form of a bright yellow solid:
UV (ethanol) λₘₐₓ=388 nm,εₘₐₓ=36 000
λₘₐₓ=301 mn,εₘₐₓ=40 120
Elemental analysis for C₃₆H₅₃N₇O₆Si₃
calculated: C 56.59 H 6.99 N 12.83 Si 11.03
found: C 56.41 H6.84N 12.81 Si 10.82

### EXAMPLE 4

Preparation of 2,4-bis(butyl 4′-diylaminobenzoate)-6-(2-ethylhexyl 4′-ylamino-α-cyanocinnamate)-s-triazine:

First step: Preparation of 2,4-bis(butyl 4′-diylaminobenzoate)-6-chloro-s-triazine:

Butyl aminobenzoate (9.94 g, 0.0515 mol) dissolved in 15 ml of dioxane is added dropwise to a solution of cyanuric chloride (9.2 g, 0.05 mol) in 100 ml of dioxane, followed by addition of 3.5 g of potassium carbonate dissolved in 15 ml of water. The reaction medium is then brought to 40° C. and a further 9.94 g of butyl aminobenzoate dissolved in 15 ml of dioxane and then 3.5 g of potassium carbonate are added. Stirring is then continued for 3 hours at 65° C. The reaction medium is filtered. 250 ml of water are added to the filtrate. The precipitate formed is filtered off by suction, Dried and recrystallized from 250 ml of toluene. 16.5 g (yield: 66%) of the expected derivative are obtained in the form of a white powder.

Second Step: Preparation of the Compound of Example 4:

The preceding derivative (10 g, 0.033 mol) and 2-ethylhexyl 3-(4-aminophenyl)-2-cyanoacrylate (16.4 g, 0.033 mol) in 100 ml of toluene are refluxed for 2 hours. After concentrating, the pasty residue is taken up in a hot 95/5 isopropanol/water mixture. The yellow precipitate obtained is dried under vacuum. 12 g (yield: 48%) of the product of Example 4 are obtained, this product having the following characteristics:
Bright yellow solid
UV (ethanol) λₘₐₓ=389 nm,εₘₐₓ=42 210
λₘₐₓ=312 nm,εₘₐₓ=64 300
Elemental analysis for C₄₃H₅1N₇O₆, 1H₂O
calculated: C 66.24 H 6.80 N 12.58 O 14.37
found: C 66.01 H 6.99 N 12.50 O 14.48

### EXAMPLE 5

Preparation of 2-(4′-ylamino-2-methanesulfonylacrylonitrile)-4-(butyl 4′-ylaminobenzoate)-6-{[1,3,3,3-tetramethyl-1-[(trimethylsilyl)oxy]-disiloxanyl]propyl-3-ylamino)-s-triazine

First Step: Preparation of 3-(4-aminophenyl)-2-methanesulfonylacrylonitrile:

A mixture of 4-aminobenzaldehyde oligomer (12 g, 0.1 mol) and methanesulfonylacetonitrile (11.9 g, 0.1 mol) dispersed in 150 ml of ethanol in the presence of a catalytic amount of diethylamine (0.125 ml) and of acetic acid (0.375 ml) is refluxed for 6 hours. An insoluble material is removed by filtration.

The filtrate is concentrated to one third of its volume under vacuum. The precipitate obtained is filtered off and dried. 11.6 g (yield: 52%) of the expected derivative are obtained in the form of an orange-coloured solid.

Second Step: Preparation of 3-[4-(4,6-dichloro[1,3,5]triazin-2-ylamino)phenyl]-2-methanesulfonylacrylonitrile:

A solution of the preceding derivative (11.1 g, 0.05 mol) in 100 ml of acetone and 100 ml of aqueous sodium bicarbonate solution (4.2 g, 0.05 mol) are sequentially added dropwise over 30 minutes to a solution of cyanuric chloride (9.2 g, 0.05 mol) in 120 ml of acetone cooled to about 5° C. The mixture is allowed to return to room temperature. The abundant precipitate formed is filtered off and washed with acetone. 10 g (yield: 54%) of the expected derivative are obtained in the form of a pale yellow powder.

Third Step: Preparation of Butyl 4-{4-chloro-6-[4-(2-cyano-2-methanesulfonylvinyl)phenylaminol][1.3,5]triazin-2-ylamino}benzoate:

The preceding derivative (6 g, 0.016 mol) is suspended in 50 ml of DMF. A solution of butyl 4-aminobenzoate (3.1 g, 0.016 mol) in 40 ml of DMF and 40 ml of aqueous sodium bicarbonate solution (1.3 g, 0.016 mol) are added sequentially. Heating is continued at 90° C. for 2 hours. The resulting mixture is cooled. The yellow precipitate formed is filtered off, washed with water, dried under vacuum and used without further purification in the following step.

Fourth Step:

A mixture of the preceding derivative (3.8 g, 0.0072 mol) and of 1-amino-3-[1,3,3,3-tetramethyl-1-[(trimethylsilyl)oxy]disiloxanyl]propane (4 g, 0.014 mol) in 50 ml of toluene is heated at 90° C. for 2 hours with stirring, while sparging with nitrogen. The toluene is stripped off under vacuum and the residue obtained is purified by chromatography on silica (eluent: 70/30 heptane/EtOAc). 1.5 g (yield: 26%) of the product of Example 5 are obtained, this product having the following characteristics:
Yellow powder
UV (ethanol) λₘₐₓ=389 nm,εₘₐₓ=43 590
λₘₐₓ=300 nm,εₘₐₓ=37 740
Elemental analysis for C₃₄H₅₁N₇O₆Si₃
calculated: C 53.03 H 6.67 N 12.73 S 4.16 Si 10.94
found: C 52.52 H 6.71 N 12.21 S 3.83 Si 10.54

### EXAMPLE 6

Preparation of 2-(ethyl 4′-ylamino-α-cyanocinnamate)-4-(butyl 4′-ylaminobenzoate)-6-(trimethylsilanylmethylylamino)-s-triazine

A mixture of ethyl 3-{4-[4-chloro-6-(4-pentanoylphenylamino)[1,3,5]triazin-2-ylamino]phenyl}-2-cyanoacrylate (1 g, 0.0019 mol) obtained in the first step of the compound of Example 2 and aminomethyltrimethylsilyl [lacuna] (0.39 g, 0.0038 mol) in 30 ml of toluene is heated at about 95° C. for 1 hour. The toluene is evaporated off under vacuum. The paste obtained is purified by chromatography twice on a column of silica (eluent: 50/50 heptane/EtOAc). 0.25 g (yield: 20%) of the final compound is obtained in the form of a bright yellow powder, the proton NMR spectrum of which is in accordance with the expected structure:
V (ethanol) λₘₐₓ=389 nm,εₘₐₓ=30 300
λₘₐₓ=303 nm,εₘₐₓ=39 820

### EXAMPLE 7

Preparation of 2,4,6-tris(isobutyl 4′-ylamino-α-cyanocinnamate)-s-triazine:

First Step: Preparation of Isobutyl 3-(4-aminophenyl)-2-cyanoacrylate:

4-Aminobenzaldehyde oligomer (36.3 g, 0.3 mol) is suspended in 250 ml of isobutanol. Isobutyl cyanoacetate (42.3 g, 0.3 mol) and a catalyst consisting of diethylamine (0.375 ml) and of acetic acid (1.12 ml) are added thereto. The mixture is refluxed for 3 hours. A light amount of insoluble material is filtered off while hot. After cooling the filtrate, the orange precipitate formed is filtered off. After drying, 52.6 g (yield: 72%) of the expected derivative are obtained in the form of an orange-coloured solid.

Second Step:

Cyanuric chloride (37 g, 0.02 mol) is suspended in toluene at a temperature of about 10° C., while sparging with nitrogen. The preceding derivative (14.6 g, 0.06 mol) is added thereto and the reaction mixture is gradually heated to about 45° C. The reaction is maintained at about 60° C. for 1 hour and is then maintained at the reflux point of the toluene for 2 hours. The hydrochloric acid formed is trapped in a wash flask containing sodium hydroxide solution. The reaction mixture is cooled and the yellow precipitate formed is filtered off and dried. 8.6 g (yield: 53%) of the derivative of Example 7 are obtained, this product having the following characteristics:
Bright yellow solid
UV (ethanol) λₘₐₓ=390 nm,εₘₐₓ=88 630
Elemental analysis for C₄₅H₄₅N₉O₆
calculated: C 66.90 H 5.61 N 15.60 O 11.88
found: C 66.35 H 5.70 N 15.43 O 11.99

### EXAMPLE 8

Preparation of 2,4,6-tris(2-ethylhexyl 4′-ylamino-α-cyanocinnamate)-s-triazine

First Step: Preparation of 2-ethylhexyl 3-(4-aminophenyl)-2-cyanoacrylate:

4-Aminobenzaldehyde oligomer (36.3 g, 0.3 mol) is suspended in 450 ml of isobutanol. 2-ethylhexyl cyanoacetate (59.1 g, 0.3 mol) and a catalyst consisting of diethylamine (0.375 ml) and of acetic acid (1.12 ml) are added thereto. The mixture is refluxed for 5 hours. A second addition of catalyst was performed and the reaction mixture refluxed for a further 4 hours. A light amount of insoluble material is filtered off while hot. After cooling the filtrate, it is concentrated to a volume of 300 ml and allowed to crystallize under cold conditions. After drying, 59.6 g (yield: 66%) of the expected derivative are obtained in the form of an orange-coloured solid.

Second Step:

Cyanuric chloride (37 g, 0.02 mol) is suspended in toluene at a temperature of about 10° C., while sparging with argon. The preceding derivative (18 g, 0.06 mol) is added thereto and the reaction mixture is gradually heated to about 45° C. The reaction is maintained at about 60° C. for 1 hour and then maintained at the reflux point of the toluene for 2 hours. The hydrochloric acid formed is trapped in a washing flask containing sodium hydroxide solution. The reaction mixture is cooled and the yellow precipitate formed is filtered off and dried. 18.8 g (yield: 96%) of the derivative of Example 8 are obtained, this product having the following characteristics:
Bright yellow solid
UV (ethanol) λₘₐₓ=398 nm,εₘₐₓ=88 630
Elemental analysis for C₅₇H₆₉N₉O₆, 1H2O
calculated: C 68.86 H 7.20 N 12.68 O 11.26
found: C 68.01 H 7.02 N 12.64 O 10.63

### EXAMPLE 9

Preparation of 2,4-bis(2-ethylhexyl 4′-ylamino-α-cyanocinnamate)-6-{[1,3,3,3-tetramethyl-1-[(trimethylsilyl)oxy]disiloxanyl]propyl-3-ylamino}-s-triazine:

Step 1: Preparation of 2,4-dichloro-6-{[1,3,3,3-tetramethyl-1-[(trimethylsilyl)oxy]disiloxanyl]propyl-3-ylamino}-s-triazine:

1-Amino-3-[1,3,3,3-tetramethyl-1-[(trimethylsilyl)oxy]disiloxanyl]propane (41.7 g, 0.149 mol) and a solution of sodium bicarbonate (11.4 g, 0.136 mol) in 120 ml of water are added dropwise to a solution of cyanuric chloride (25 g, 0.136 mol) in 250 ml of acetone at 0° C., such that the pH is between 3 and 5. At the end of the introduction, the pH is 6.5. Stirring is then continued for 1 hour 30 minutes at 10° C. and the mixture is then allowed to warm to the laboratory temperature. The reaction medium is filtered. An oil separates out by settling. The acetone is evaporated off under vacuum. The lower phase is recovered and left to crystallize.

After drying, 55.2 g (yield: 95%) of the expected derivative are obtained in the form of a white powder (melting point: 59° C.)

Second Step:

The preceding derivative (2 g, 0.00468 mol) is dissolved in 50 ml of toluene. The 2-ethylhexyl 3-(4-aminophenyl)-2-cyanoacrylate obtained in the first step of derivative 7 (2.81 g, 0.00935 mol) is added thereto and the mixture is heated at 95° C. for 5 hours. The solvent is evaporated off and the gum obtained is purified by passing it through a column of silica (eluent: 80/20 heptane/EtOAc). 2.47 g (yield: 55%) of the derivative of Example 9 are obtained, this product having the following characteristics:
Proton NMR spectrum in accordance with the expected structure
Bright yellow solid
UV (ethanol) λₘₐₓ=400 nm,εₘₐₓ=76 820

### EXAMPLE 10

Water-in-silicone Emulsion Containing a Non-flavonoid Polyphenol Derivative: Curcumin

| | Amounts in | |
|---|---|---|
| Ingredients | grams | Phase |
| Oxyethylene oxypropylene | 10 | A |
| polydimethyl/methylsiloxane (396/4) | | |
| (18/18 OE/OP) as a 10% solution in D5 | | |
| (DC2-5225C - Dow Corning) | | |
| Cyclopentadimethylsiloxane | 12.5 | |
| (DC 245 Fluid - Dow Corning) | | |
| Mixture of natural tocopherols/soybean oil | 0.1 | |
| (D Mixed Tocopherols 50% Oil - Bizen) | | |
| Preserving agents | qs | |
| Curcumin (Sigma) | 0.05 | C |
| Propylene glycol | 27 | |
| Demineralized water | qs 100 g | B |

Procedure

The constituents of phase A are introduced into the final beaker and homogenized by stirring without heating. Phase B is weighed out in a separate beaker and then poured into phase A with stirring. Stirring is continued for 10 minutes. The constituents of phase C are introduced. The mixture is homogenized and stirring is continued until a gel is obtained.

### EXAMPLE 11

Water-in-oil Emulsion Containing Iron Oxides

| | Amounts in | |
|---|---|---|
| Ingredients | grams | Phase |
| Polyethylene glycol dipolyhydroxystearate (30 EO) | 2 | A |
| (Arlacel P 135 - Uniqema) | | |
| Oxyethylene oxypropylene polymethyllauryl/- | 2 | |
| methylsiloxane (35/3) | | |
| (18 OE/18 OP) (MW: 25 000) (Q2-5 200 - Dow | | |
| Corning) | | |
| C₁₂/C₁₅ alkylbenzoate | 15 | |
| (Finsolv TN - Witco) | | |
| Drometrizole trisiloxane screening agent | 2 | |
| (Silatrizole - Rhodia Chimie) | | |
| Preserving agents | qs | |
| Polydimethylsiloxane | 3 | C |
| (DC200 Fluid - 1000 cst - Dow Corning) | | |
| Coated nanoiron oxides | 2 | |
| Glycerol | 5 | B |
| Demineralized water | qs 100 g | |

Procedure

The constituents of phase A are weighed out in the final beaker. The constituents of phase B are weighed out in a separate beaker. Phases A and B are heated to about 80° C. Phase A is stirred and phase B is introduced slowly. Stirring is continued for 10 minutes. Phase C is added. The mixture is homogenized. Stirring is stopped after a smooth, shiny emulsion is obtained.

### EXAMPLE 12

Oil-in-water Emulsion Containing a Triazine Derivative of Formula (III)

| | Amounts in | |
|---|---|---|
| Ingredients | grams | Phase |
| Glyceryl mono/distearate/polyethylene glycol | 2 | A¹ |
| stearate mixture (100 EO) | | |
| (Arlacel 165 FL - ICI) | | |
| Stearyl alcohol (Lanette 18 - Cognis) | 1 | |
| Stearic acid from palm oil | 1.5 | |
| (Stearine TP - Stéarinerie Dubois) | | |
| Polydimethylsiloxane | 0.5 | |
| (Dow Corning 200 fluid 250 cs - Dow Corning) | | |
| C₁₂/C₁₅ alkylbenzoate | 10 | |
| (Finsolv TN - Witco) | | |
| Preserving agents | qs | |
| 2,4,6-Tris(2-ethylhexyl 4′-ylamino-α- | 2 | A² |
| cyanocinnamate)-s-triazine | | |
| (compound of Example 8) | | |
| 2-Ethylhexyl p-dimethylaminobenzoate | 4 | |
| (Escalol 507 - ISP) | | |
| 2,2,4,4,6,6,8-heptamethylnonane | 4 | C |
| (Permethyl 101A - General Bayer Silicones) | | |
| Crosslinked acrylic acid/C₁₀/C₃₀ alkyl acrylate | 0.2 | |
| copolymer (Pemulen TR-1 - Noveon) | | |
| Hydroxypropylmethylcellulose | 0.1 | |
| (Methocel F 4M - Dow Chemical) | | |
| Triethanolamine | qs pH7 | D |
| Glycerol | 5 | B |
| Demineralized water | qs 100 g | |

Procedure

Phase A² is homogenized using a Retsch MM200 mill under the following conditions: 2 balls 1.5 cm in diameter, stirring=20 minutes, frequency 30. Phase A¹ is introduced into the final beaker. Phase A²is added. Phase B is weighed out in a separate beaker. Phases (A¹+A ²) and B are heated at 80° C. Phase B is stirred and phases (A¹+A²) are added. The mixture is stirred for 10 minutes. At about 50° C., phase C is added. This mixture is homogenized and phase D is introduced. The resulting mixture is allowed to cool to 25° C.

### EXAMPLE 13

Oil-in-water Emulsion Containing a Quinone Compound: Lawsone

| | Amounts in | |
|---|---|---|
| Ingredients | grams | Phase |
| Glyceryl mono/distearate/polyethylene glycol | 2 | A |
| stearate mixture (100 EO) | | |
| (Arlacel 165 FL - ICI | | |
| Stearyl alcohol (Lanette 18 - Cognis) | 1 | |
| Stearic acid from palm oil | 1.5 | |
| (Stearine TP - Stéarinerie Dubois) | | |
| C₁₂/C₁₅ alkyl benzoate | 10.5 | |
| (Finsolv TN - Witco) | | |
| Drometrizole trisiloxane screening agent | 2 | |
| (Silatrizole - Rhodia Chimie) | | |
| Preserving agents | qs | |
| 2,2,4,4,6,6,8-heptamethylnonane | 4 | C |
| (Permethyl 101A - General Bayer Silicones) | | |
| Crosslinked acrylic acid/C₁₀/C₃₀ alkyl acrylate | 0.2 | |
| copolymer (Pemulen TR-1 - Noveon) | | |
| Hydroxypropylmethylcellulose | 0.1 | |
| (Methocel F 4M - Dow Chemical) | | |
| Lawsone (Aldrich) | 1 | D |
| Triethanolamine | qs pH 7 | |
| Demineralized water | 4 | |
| Glycerol | 5 | B |
| Demineralized water | qs 100 g | |

Procedure

Phase A is weighed out in the final beaker. Phase B is weighed out in a separate beaker. Phases A and B are heated until dissolution is complete (80° C). Phase B is stirred and phase A is poured in. Stirring is continued for 10 minutes. At about 50° C., phase C is added. The mixture is homogenized and the predissolved phase D is introduced. The resulting mixture is homogenized and allowed to cool to 25° C.

### EXAMPLE 14

Oil-in-water Emulsion Containing an Azo Compound: Tartrazine

| | Amounts in | |
|---|---|---|
| Ingredients | grams | Phase |
| Glyceryl mono/distearate/polyethylene glycol | 2 | A |
| stearate mixture (100 EO) | | |
| (Arlacel 165 FL - ICI) | | |
| Stearyl alcohol (Lanette 18 - Cognis) | 1 | |
| Stearic acid from palm oil | 1.5 | |
| (Stearine TP - Stéarinerie Dubois) | | |
| C₁₂ /C₁₅ alkyl benzoate | 10.5 | |
| (Finsolv TN - Witco) | | |
| Drometrizole trisiloxane screening agent | 2 | |
| (Silatrizole - Rhodia Chimie) | | |
| Preserving agents | qs | |
| 2,2,4,4,6,6,8-heptamethylnonane | 4 | C |
| (Permethyl 101A - General Bayer Silicones) | | |
| Crosslinked acrylic acid/C₁₀/C₃₀ alkyl acrylate | 0.2 | |
| copolymer (Pemulen TR-1 - Noveon) | | |
| Hydroxypropylmethylcellulose | 0.1 | |
| (Methocel F 4M - Dow Chemical) | | |
| Tartrazine | 4 | |
| Triethanolamine | qs pH 7 | |
| Demineralized water | 12.5 | |
| Glycerol | 5 | B |
| Demineralized water | qs 100 g | |

Procedure

Phase A is weighed out in the final beaker. Phase B is weighed out in a separate beaker. Phases A and B are heated until dissolution is complete (80° C.). Phase B is stirred and phase A is poured in. Stirring is continued for 10 minutes. At about 50° C., phase C is added. The mixture is homogenized and the predissolved phase D is introduced. The resulting mixture is homogenized and allowed to cool to 25° C.

### EXAMPLE 15

Oil-in-water Emulsion Containing a Flavonoid Polyphenol: Luteolin

| | Amounts in | |
|---|---|---|
| Ingredients | grams | Phase |
| Glyceryl mono/distearate/polyethylene glycol | 2 | |
| stearate mixture (100 EO) | | |
| (Arlacel 165 FL - ICI) | | |
| Stearyl alcohol (Lanette 18 - Cognis) | 1 | |
| Stearic acid from palm oil | 1.5 | |
| (Stearine TP - Stéarinerie Dubois) | | |
| Polydimethylsiloxane | 0.5 | |
| (Dow Corning 200 fluid 250 cst - Dow Corning | | |
| C₁₂/C₁₅ alkyl benzoate | 105 | |
| (Finsolv TN - Witco) | | |
| Drometrizole trisiloxane screening agent | 2 | |
| (Silatrizole - Rhodia Chimie) | | |
| Preserving agents | qs | |
| 2,2,4,4,6,6,8-heptamethylnonane | 4 | C |
| (Permethyl 101A - General Bayer Silicones) | | |
| Crosslinked acrylic acid/C₁₀/C₃₀ alkyl acrylate | 0.2 | |
| copolymer (Pemulen TR-1 - Noveon) | | |
| Hydroxypropylmethylcellulose | 0.1 | |
| (Methocel F 4M - Dow Chemical) | | |
| Luteolin (Extrasynthese) | 1 | D |
| Triethanolamine | qs pH 7 | |
| Demineralized water | 5 | |
| Glycerol | 5 | B |
| Demineralized water | qs 100 g | |

Procedure

Phase A is weighed out in the final beaker. Phase B is weighed out in a separate beaker. Phases A and B are heated until dissolution is complete (80° C.). Phase B is stirred and phase A is poured in. Stirring is continued for 10 minutes. At about 50° C., phase C is added. The mixture is homogenized and the predissolved phase D is introduced. The resulting mixture is homogenized and allowed to cool to 25° C.

Evaluation of the Protective Effect of the Compositions According to the Invention of the Endogenous Carotenoids under Exposure to Light Radiation with a Wavelength Ranging from 370 to 500 nm

This evaluation is performed by means of a β-carotene decolorization test, based on the principle that β-carotene becomes degraded when irradiated with the blue component of sunlight. This degradation is reflected by decolorization, which is monitored using a Minolta chromameter by means of its component b*.

Experimental Conditions

The exposures to blue light are performed using the Sun-Test CPS+ machine from the company Heraeus at 30 J/cm² (expressed as applied UVA).

An extemporaneous preparation of a 0.05% solution of β-carotene in Miglyol 812 is prepared. 2 drops of the solution are spread onto a filter paper (inside an eyelet); the paper is left to dry at room temperature and in the absence of light.

The test formulations are spread at a rate of 2 mg/cm² onto quartz plates (5 cm×5 cm) and left to dry for 30 minutes at room temperature and in the absence of light. The quartz plate containing the formula is placed on the filter paper impregnated with β-carotene and the assembly is irradiated using the Sun-Test CPS+ machine.

The b* values are recorded using a Minolta chromameter after an exposure of 30 J/cm² measured in UVA.

Compositions Tested

Each of the compositions of Examples 12, 13, 14 and 15 as defined above containing, respectively, as agent for screening out radiation with a wavelength ranging from 370 to 500 nm: a triazine derivative of formula (III), lawsone, tartrazine and luteolin, is compared with a placebo composition of identical support not containing any screening agent.

Results

The percentage of improvement of the stability of the β-carotene with respect to the decolorization induced by light between 400 and 450 nm obtained by each test formulation relative to the same placebo formulation not containing any agent for screening out the said light is calculated.

The results obtained are indicated in the table below.

TABLE 1

| | % improvement in the stability of β-carotene with |
|---|---|
| | respect to the decolorization induced by light |
| Test composition | between 400 and 450 nm |
| Example 12 | +40% |
| (aminoarylvinyl-s- | |
| triazine compound) | |
| Example 13 | +10.3% |
| (lawsone) | |
| Example 14 | +41% |
| (tartrazine) | |
| Example 15 | +40% |
| (luteolin) | |

The above written description of the invention provides a manner and process of making and using it such that any person skilled in this art is enabled to make and use the same, this enablement being provided in particular for the subject matter of the appended claims, which make up a part of the original description and including the use of at least one agent for screening out light radiation with a wavelength ranging from 370 to 500 nm, in the manufacture of a composition applied to the surface of the skin as an agent for inhibiting the degradation of the endogenous carotenoids present in the skin. Similarly enabled are aminoarylvinyl-s-triazine compounds corresponding to formula (III) below: 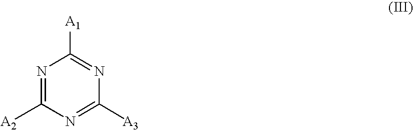
in which:
The groups A₁, A₂ and A₃, which may be identical or different, are selected from the group consisting of the groups of formulae (IV) to (VIII) below, it being understood that at least one group of formulae (IV) to (VII) is present: 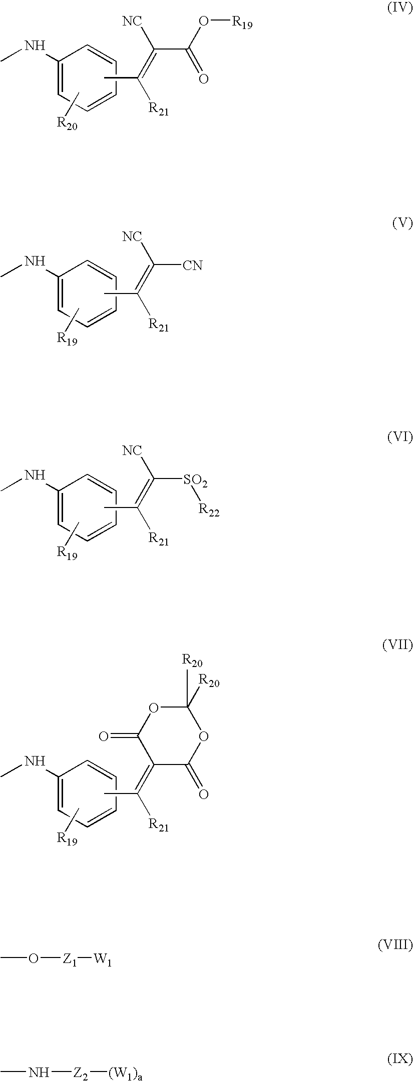
in which:
R₂₀ represents hydrogen, a C₁-C₄ alkyl radical or a C₁-C₄ alkoxy radical,
R₁₉ represents a linear or branched C₁-C₂₀ alkyl radical, a linear or branched C₂-C₂₀ hydroxyalkyl radical or a linear or branched C₁-C₂₀ alkoxy radical,
R₂, represents hydrogen, a methyl radical or a phenyl radical optionally substituted with a C₁-C₄ alkyl radical or a C₁-C₄ alkoxy radical,
R₂₂ represents a linear or branched C₁-C₂₀ alkyl radical or a phenyl radical optionally substituted with a C₁-C₄ alkyl radical or a C₁-C₄ alkoxy radical,
Z₁ represents a divalent radical providing the bond between —O— and —W₁,
Z₂ represents a divalent radical when a=1 and a trivalent radical when a=2, providing the bond between —NH— and —(W₁)a,
Z₁ and Z₂ possibly being C₁-C₁₂ alkylene, optionally substituted with one or more hydroxyl groups and possibly containing one or more oxygen atoms or one or more amino groups and optionally containing a double bond, W₁ represents:
(i) either a silicone radical comprising at least one unit of formula (X) below: 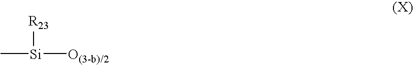
   in which R₂₃ denotes a saturated or unsaturated, linear or branched C₁-C₃₀ hydrocarbon-based group; a C₁-C₈ halohydrocarbon group; a phenyl radical; a 3,3,3-trifluoropropyl radical or a trimethylsilyloxy group, and b is equal to 1 or 2, (ii) or a radical of formula (XI) below:
   —SiR₂₄R₂₅R₂₆ (XI)
   in which R₂₄, R₂₅ and R₂₆, which may be identical or different, are selected from the group consisting of linear or branched C₁-C₈ alkyl and alkenyl radicals; the groups A₁, A₂ and A₃ may also represent a UVB- and/or UVA-absorbing chromophore preferably selected from the group consisting of aryl groups substituted with hydroxyl groups, linear or branched C₁-C₂₀ alkyl groups and linear or branched C₁-C₂₀ alkoxy groups; aminobenzylidenecamphor groups; aminobenzotriazole groups; linear or branched aminobenzoate, aminobenzalmalonate, aminosalicylate, C₁-C₂₀ aminocinnamate or anthranilate ester groups;
   with the proviso that:
the three groups A₁, A₂ and A₃ do not denote the same formula (IV),
when A₂ and A₃ denote a group of formula (IV), then A₁ does not denote a group of formula (VIII) or (IX), and compositions containing at least one of these compounds.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

As used herein, where a polymer is noted as being “obtained from” or “comprising”, etc. one or more monomers (or monomer units) this description is of the finished polymer material itself and the repeating units therein that make up, in whole or part, this finished product. One of ordinary skill in the art understands that, speaking precisely, a polymer does not include individual, unreacted and reactive “monomers,” but instead is made up of repeating units derived from reacted monomers.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A method of conserving the endogenous reserve of skin carotenoids comprising applying to the surface of skin in need thereof an endogenous reserve-conserving effective amount of at least one agent for screening out light radiation with a wavelength ranging from 370 to 500 nm.

2. The method according to claim 1, in which the agent for screening out light radiation with a wavelength ranging from 370 to 500 nm comprises a material selected from the group consisting of:
(i) flavonoid polyphenols, non-flavonoid polyphenols and the ester, ether, heteroside and polymeric derivatives thereof;
(ii) carotenoids and β-laines;
(iii) chlorophylls;
(iv) melanins;
(v) carbohydrates;
(vi) yellow or orange-yellow mineral pigments;
(vii) azo or quinone compounds;
(viii) nitrobenzene-based dyes;
(ix) aryl vinylene ketone-based compounds;
(x) aminoarylvinyl-s-triazine compounds;
and mixtures thereof.

3. The method according to claim 1, in which the agent for screening out light radiation with a wavelength ranging from 370 to 500 nm has no antioxidant activity.

4. The method according to claim 3, in which the agent for screening out light radiation with a wavelength ranging from 370 to 500 nm is selected from the group consisting of:
(v) carbohydrates;
(vi) yellow or orange-yellow mineral pigments;
(vii) azo or quinone compounds;
(viii) nitrobenzene-based dyes;
(ix) aryl vinylene ketone-based compounds;
(x) aminoarylvinyl-s-triazine compounds;
and mixtures thereof.

5. The method according to claim 2, said agent comprising a flavonoid polyphenol selected from the group consisting of natural yellow or orange-yellow dyes of the flavone type; of the flavonol type; of the isoflavone type; of the chalcone type; of the aurone type; proanthocyanidine oligomers; hydrolysable and non-hydrolysable condensed tannin polymers, and mixtures thereof.

6. The method according to claim 2, said agent comprising a non-flavonoid polyphenol selected from the group consisting of natural or synthetic yellow or orange-yellow dyes of the flavin or curcumin type, and mixtures thereof.

7. The method according to claim 2, said agent comprising a polyphenol of polymeric form.

8. The method according to claim 7, in which the polyphenol is selected from the group consisting of proanthocyanidine oligomers and hydrolysable and non-hydrolysable condensed tannin polymers, and mixtures thereof.

9. The method according to claim 8, in which the polyphenols are selected from the group consisting of procyanidin B2 from apple, tannic acid and catechin polymers extracted from grape, tea, coffee, cocoa, chicory, onion or yellow apple peel, and mixtures thereof.

10. The method according to claim 2, said agent comprising a carotenoid selected from the group consisting of bixin, crocetin, crocin, lutein, zeoaxanthin, astaxanthin, canthaxanthin, capsanthin, cryptoxanthin, rhodoxanthin, rubixanthin, and mixtures thereof.

11. The method according to claim 2, said agent comprising a β-laine selected from the group consisting of vulgaxanthins I and II, and mixtures thereof.

12. The method according to claim 2, said agent comprising a chlorophyll selected from the group consisting of chlorophylls a, b, c and d and the acid or alkaline hydrolysates thereof, and mixtures thereof.

13. The method according to claim 2, said agent comprising a carbohydrate selected from the group consisting of yellow-coloured products derived from the heating or oxidation of monosaccharides or polysaccharides, and mixtures thereof.

14. The method according to claim 13, in which the carbohydrate is caramel.

15. The method according to claim 2, said agent comprising a melanin selected from the group consisting of pheomelanin-enriched melanins and mixtures thereof.

16. The method according to claim 2, said agent comprising a yellow or orange pigment selected from the group consisting of nanopigments with an elemental particle size of less than 100 nm and mixtures thereof.

17. The method according to claim 2, said agent comprising a quinone compound selected from the group consisting of yellow or orange-yellow naphthoquinones and the oxidation products thereof; anthraquinones and the yellow or orange-yellow oxidation products thereof, and mixtures thereof.

18. The method according to claim 17, said agent comprising a quinone compounds selected from the group consisting of juglone, lawsone and mixtures thereof.

19. The method according to claim 2, said agent comprising tartrazine.

20. The method according to claim 2, said agent comprising a compound selected from the group consisting of those of formula (1), (2) or (3), and mixtures thereof: 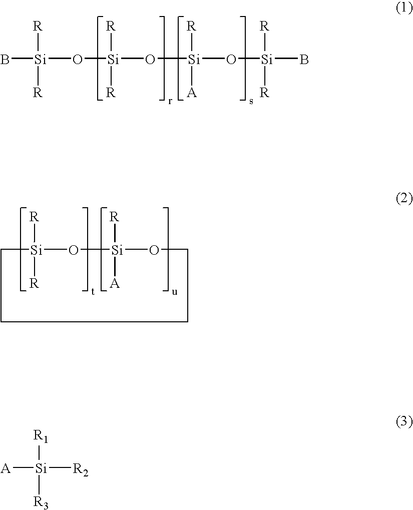
in which, for formulae (1) and (2):
R, which may be identical or different, are selected from the group consisting of C₁-C₁₀ alkyl, phenyl and 3,3,3-trifluoropropyl radicals, at least 80%, in numerical terms, of the radicals R being methyl,
B, which may be identical or different, are selected from the group consisting of the radicals R above and the radical A defined below,
r is an integer between 0 and 50 inclusive, and s is an integer between 0 and 20 inclusive, with the condition that if s is 0, then at least one of the two symbols B denotes A,
u is an integer between 1 and 6 inclusive, and
t is an integer between 0 and 10 inclusive, it being understood that t+u is greater than or equal to 3,
R₁, R₂ and R₃, which may be identical or different, are selected from the group consisting of saturated or unsaturated, linear or branched C₁-C₈ alkyl and alkenyl radicals,
the symbols A, which may be identical or different, denote a radical of formula (4a), (4b), (4c) or (4d) below: 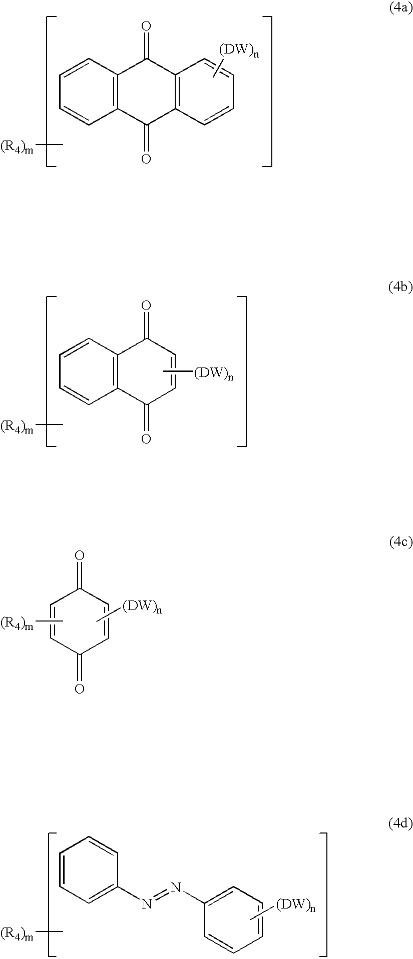
R₄, which may be identical or different, represent a linear or branched C₁-C₆ alkyl radical, an OH, C₁-C₄ alkoxy, hydroxy(C₁-C₄)alkyl, COOH, CONH₂, CN, SO₃H, halogen or NO₂ radical, a radical NR₅R₆ in which R₅ and R₆, which may be identical or different, denote a hydrogen atom or a C₁-C₈ alkyl, hydroxy(C₁-C₄)alkyl or amino(C₁-C₄)alkyl radical, or form, together with the nitrogen atom to which they are attached, a 5- or 6-membered heterocycle optionally interrupted with an oxygen or sulfur atom,
m is an integer between 0 and 2 inclusive,
n is an integer equal to 1 or 2,
D is an —SO₂NH—, —CONH— or —O— radical or a radical —NR₇— in which R₇ is H or CH₃,
W is a divalent radical of formula (5): 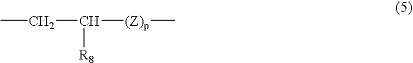
or of formula (6):
—HC═CH—(Z)ₚ— (6)
in which
R₈ denotes a hydrogen atom, a hydroxyl radical or a linear or branched, saturated or unsaturated C₁-C8 alkyl radical,
Z is a linear or branched C₁-C₆ alkylene radical optionally substituted with an OH radical or a linear or branched, saturated or unsaturated C₂-C₈ alkoxy radical,
p is an integer equal to 0 or 1.

21. The method according to claim 20, in which the compound of formula (1), (2) or (3) is selected from the group consisting of the following compounds:
4-(4-dimethylaminophenylazo)-N-[3-[1,3,3,3-tetramethyl-1-[(trimethylsilyl)oxy]disiloxanyl]propyl]benzenesulfonamide;
4-(4-dimethylaminophenylazo)-N-[3-[1,3,3,3-tetramethyl-1-[(trimethylsilyl)oxy]disiloxanyl]propyl]benzamide;
2-(4-methoxy-2-nitrophenylazo)-5-(3-trimethylsilanylpropoxy)phenol;
2,5-bis-[3-[1,3,3,3-tetramethyl-1-[(trimethylsilyl)oxy]disiloxanyl]-propylamino]benzoquinone;
2-chloro-3-[3-[1,3,3,3-tetramethyl-1-[(trimethylsilyl)-oxy]disiloxanyl]propylamino]-[1,4]-naphthoquinone;
2-[3-[1,3,3,3-tetramethyl-1-[(trimethylsilyl)oxy]disiloxanyl]propylamino]-[1,4]-naphthoquinone;
1-hydroxy-4-[3-[1,3,3,3-tetramethyl-1-[(trimethylsilyl)-oxy]disiloxanyl]propylamino]anthraquinone;
1,4-bis[3-[1,3,3,3-tetramethyl-1-[(trimethylsilyl)oxy]disiloxanyl]-propylamino]anthraquinone.

22. The method according to claim 2, said agent comprising a nitrobenzene compound selected from the group consisting of those of formula (15) or (16) and mixtures thereof: 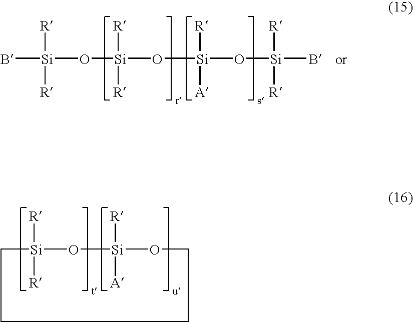
in which:
R′, which may be identical or different, are selected from the group consisting of linear or branched C₁-C₁₀ alkyl, phenyl and 3,3,3-trifluoropropyl radicals, at least 80% in numerical terms of the radicals R′ being methyl,
B′, which may be identical or different, are selected from the group consisting of the radicals R′ above and the radical A defined below,
r′ is an integer between 0 and 50 inclusive and s′ is an integer between 0 and 20 inclusive, with the condition that if s′ is zero then at least one of the two symbols B denotes A,
u′ is an integer between 1 and 6 inclusive, and t′ is an integer between 0 and 10 inclusive, it being understood that t′+u′ is greater than or equal to 3,
and the symbol A′ denotes a monovalent radical directly linked to a silicon atom, and which corresponds to formula (17) below: 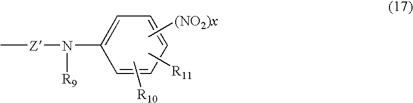
Z′ is a divalent radical: 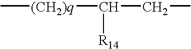
or hydrogen,
x is 1 or 2,
q represents an integer between 0 and 10 inclusive,
R₉ represents hydrogen or a C₁-C₄ alkyl radical,
R₁₀ represents hydrogen or a C₁-C₄ alkyl radical, or the divalent radical Z′ defined above,
R₁₁ represents hydrogen or a radical NR₁₂R₁₃ in which R₁₂ and R₁₃ represent hydrogen or a C₁-C₄ alkyl radical, a C₂-C₄ monohydroxyalkyl or dihydroxyalkyl radical or a divalent radical Z, it being understood that at least one radical Z′
R₁₄ represents hydrogen, an OH or halogen radical, a C₁-C₄ alkyl radical or a C₁-C₄ alkoxy radical.

23. The method according to claim 22, in which the compound of formula (15) or (16) is selected from the group consisting of the following compounds:

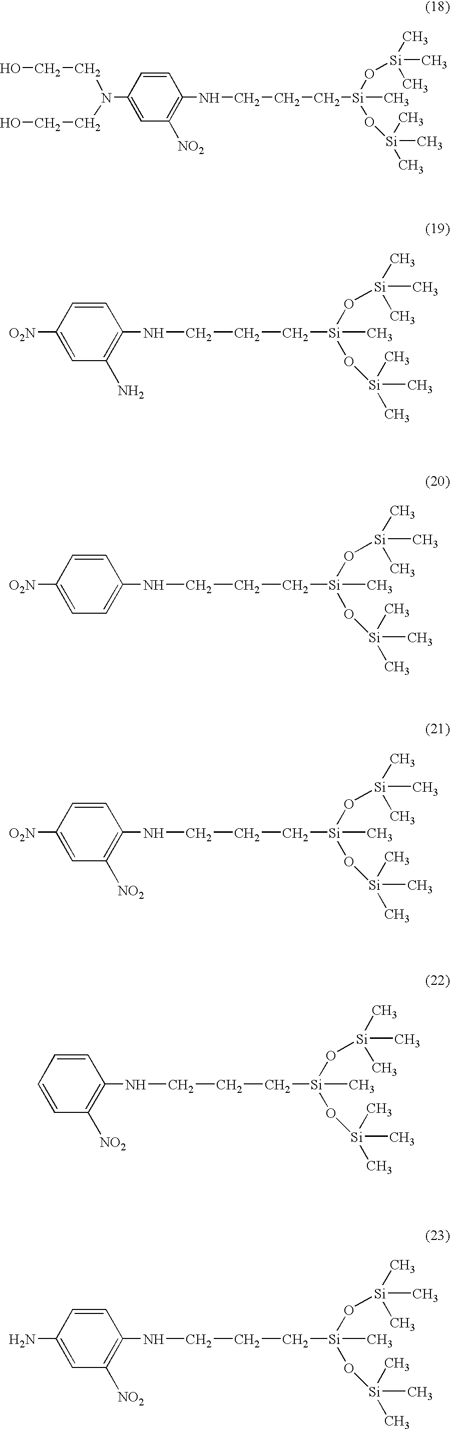

24. The method according to claim 2, said agent comprising an aryl vinyl ketone compound selected from the group consisting of those of formulae (I) and (II) below, and mixtures thereof: 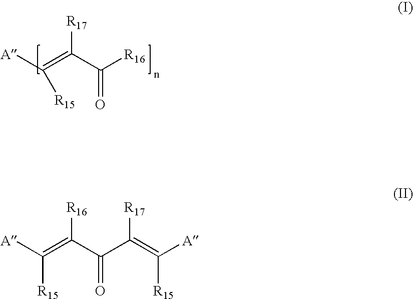
in which:
n=1 or 2,
A″, in formula (I) when n=1 or in formula (II), is an aryl radical selected from the group consisting of formulae (a) to (d) below, or in formula (I) when n=2, is a radical selected from the group consisting of formulae (e) to (h) below: 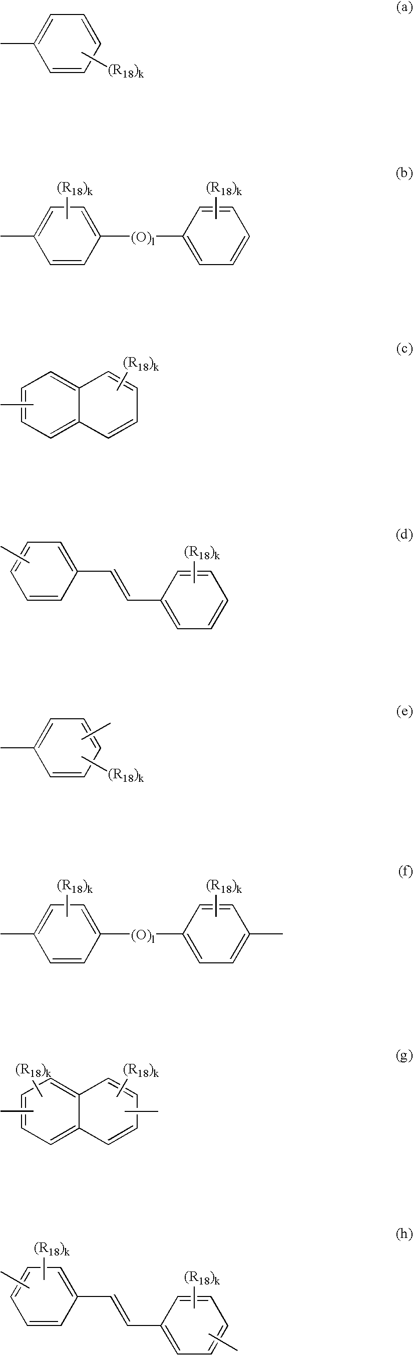
in which:
each of the symbols R₁₈ independently represents an OH group, a halogen atom, a linear or branched C₁₋₆ alkyl group optionally containing a silicon atom or a siloxane group, a linear or branched C₁₋₆ alkoxy group optionally containing a silicon atom or a siloxane group, a linear or branched C₁₋₅ alkoxycarbonyl group, or a linear or branched C₁₋₆ alkylsulfonamide group optionally containing a silicon atom, a siloxane group or an amino acid function,
k represents an integer between 0 and 3 inclusive,
1 represents 0 or 1,
R₁₅ represents hydrogen or an OH group,
R₁₆ represents hydrogen, a linear or branched C₁₋₆ alkyl group optionally containing a silicon atom or a siloxane group, a cyano group, a C₁₋₆ alkylsulfonyl group or a phenylsulfonyl group,
R₁₇ represents a linear or branched C₁₋₆ alkyl group optionally containing a silicon atom, a siloxane group or a phenyl group that can form a bicycle and optionally substituted with one or two radicals R₁₈ as defined above,
R₁₆ and R₁₇ may together form a monocyclic, bicyclic or tricyclic C₂₋₁₀ hydrocarbon-based residue, optionally interrupted with one or more nitrogen, sulfur and oxygen atoms and possibly containing another carbonyl, and optionally substituted with a linear or branched C₁-C₈ alkylsulfonamide group, optionally containing a silicon atom, a siloxane group or an amino acid function; on condition that when n=1, R₁₆ and R₁₇ do not form a camphor nucleus.

25. The method according to claim 24, in which the agent comprises at least one compound of formula (I) selected from the group consisting of:
1-(2,4,5-trimethoxyphenyl)-4,4-dimethylpent-1-en-3-one: 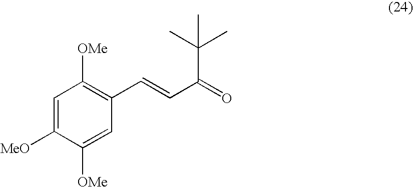
3-(3-ethoxy-4-butoxybenzylidene)-2,3,4a,8a-tetrahydrochromen-4-one: 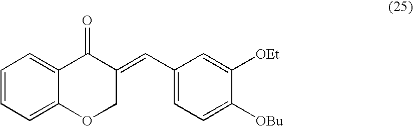
3-(4-methoxybenzylidene)-2,3,4a,8a-tetrahydrochromene-4-thione: 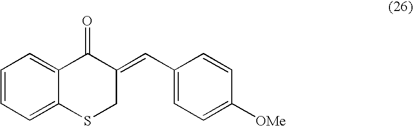
2-(3-methoxy-4-butoxybenzylidene)-indan-1-one: 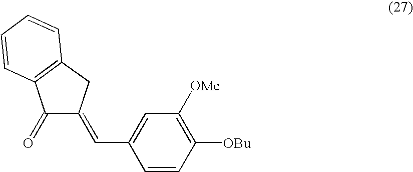
2-(3-methoxy-4-butoxybenzylidene)-3,4-dihydro-2H-naphthalen-1-one: 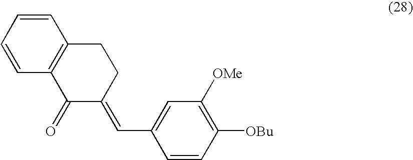
2-benzylidenebenzofuran-3-one: 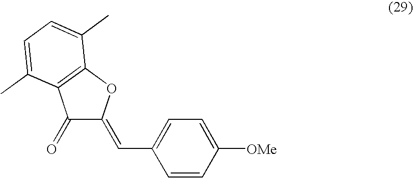
2-(3,5-dimethoxy-4-hydroxybenzylidene)indan-1,3-dione: 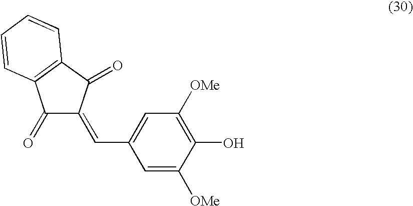
2-benzylidenebenzo[b]thiophen-3-one: 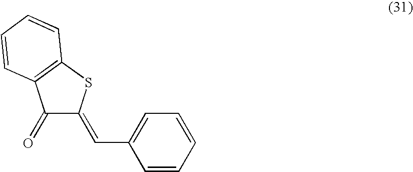
4-(3-methoxy-4-butoxybenzylidene)-5-methyl-2-phenyl-2,4-dihydropyrazol-3-one: 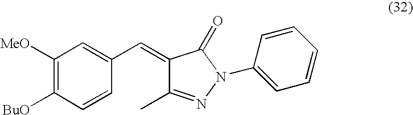
5-(3-methoxy-4-butoxybenzylidene)-2-phenyl-3,5-dihydroimidazol-4-one: 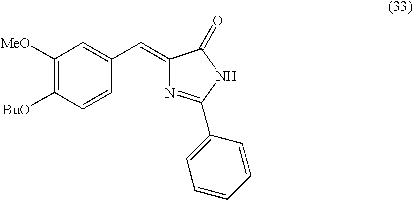
1-(2-hydroxy-4-methoxyphenyl)-3-(4′-methoxyphenyl)propenone: 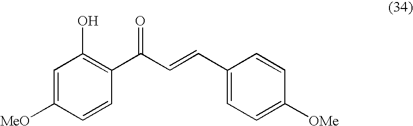
3-hydroxy-1-(2-hydroxy-4-methoxyphenyl)-3-phenylpropenone: 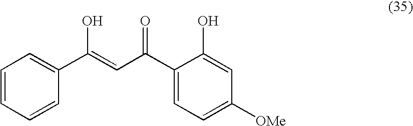
the para-xylidene compound below: 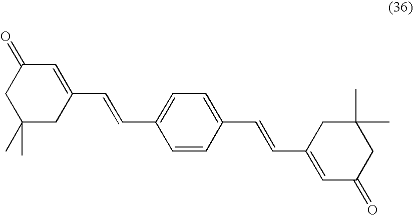
2,5-dimethoxyphenylene-1,4-bis(3-methylidenebicyclo[2.2.1]heptan-2-one): 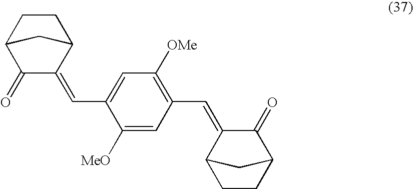
2,5-dihexyloxyphenylene-1,4-bis(3-methylidene-1,7,7-trimethylbicyclo[2.2.1]heptan-2-one):

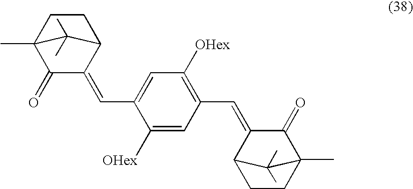

26. The method according to claim 24, in which the agent comprises at least one compound of formula (II) selected from the group consisting of:
2,5-bis(4-hexyloxybenzylidene)cyclopentanone;
2,5-bis[4-(3-trimethylsilanylpropyloxy)benzylidene]cyclopentanone.

27. The method according to claim 2, said agent comprising an aminoarylvinyl-s-triazine compound corresponding to formula (III) below: 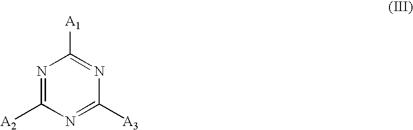
in which:
the groups A₁, A₂ and A₃, which may be identical or different, are selected from the group consisting of the groups of formulae (IV) to (VIII) below, it being understood that at least one group of formulae (IV) to (VII) is present: 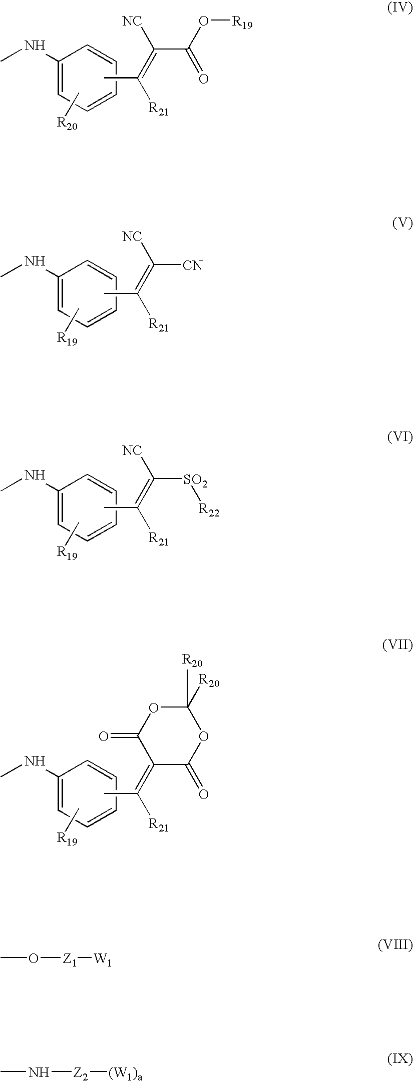
in which:
R₂₀ represents hydrogen, a C₁-C₄ alkyl radical or a C₁-C₄ alkoxy radical,
R₁₉ represents a linear or branched C₁-C₂₀ alkyl radical, a linear or branched C₂-C₂₀ hydroxyalkyl radical or a linear or branched C₁-C₂₀ alkoxy radical,
R₂₁ represents hydrogen, a methyl radical or a phenyl radical optionally substituted with a C₁-C₄ alkyl radical or a C₁-C₄ alkoxy radical,
R₂₂ represents a linear or branched C₁-C₂₀ alkyl radical or a phenyl radical optionally substituted with a C₁-C₄ alkyl radical or a C₁-C₄ alkoxy radical,
Z₁ represents a divalent radical providing the bond between —O— and —W₁,
Z₂ represents a divalent radical when a=1 and a trivalent radical when a=2, providing the bond between —NH— and —(W₁)a,
Z₁ and Z₂ possibly being C₁-C₁₂ alkylene, optionally substituted with one or more hydroxyl groups and possibly containing one or more oxygen atoms or one or more amino groups and optionally containing a double bond,
W₁ represents:
(i) either a silicone radical comprising at least one unit of formula (X) below 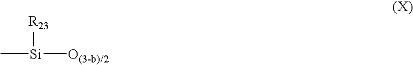
in which R₂₃ denotes a saturated or unsaturated, linear or branched C₁-C₃₀ hydrocarbon-based group; a C₁-C₈ halohydrocarbon group; a phenyl radical; a 3,3,3-trifluoropropyl radical or a trimethylsilyloxy group, and b is equal to 1 or 2, (ii) or a radical of formula (XI) below:
—SiR₂₄R₂₅R₂₆ (XI)
in which R₂₄, R₂₅ and R₂₆, which may be identical or different, are selected from the group consisting of linear or branched C₁-C₈ alkyl and alkenyl radicals; the groups A₁, A₂ and A₃ may also represent a UVB- and/or UVA-absorbing chromophore preferably selected from the group consisting of aryl groups substituted with hydroxyl groups, linear or branched C₁-C₂₀ alkyl groups and linear or branched C₁-C₂₀ alkoxy groups; aminobenzylidenecamphor groups; aminobenzotriazole groups; linear or branched aminobenzoate, aminobenzalmalonate, aminosalicylate, C₁-C₂₀ aminocinnamate or anthranilate ester groups.

28. Use according to claim 27, in which the compounds of formula (III) are selected from the group consisting of those for which W is a silicone radical corresponding to one of the three formulae (XII) to (XIV) below: 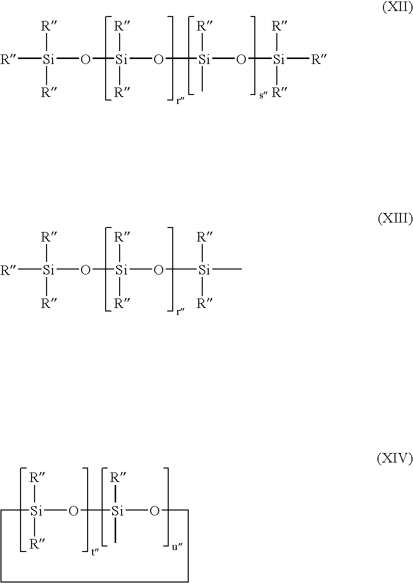
in which:
R″, which may be identical or different, are selected from the group consisting of linear or branched, saturated or unsaturated C₁-C₂₀ alkyl radicals, a phenyl radical and a 3,3,3-trifluoropropyl radical, at least 80% in numerical terms of the radicals R being methyl radicals,
r″ is an integer chosen between 0 and 50 inclusive,
s″ is an integer chosen between 0 and 20 inclusive,
u″ is an integer between 1 and 6 inclusive,
t″ is an integer between 0 and 10 inclusive,
t″+u″ is greater than or equal to 3.

29. The method according to claim 28, wherein said agent comprises at least one compound of formula (III) selected from the group consisting of those for which W is a silicone radical corresponding to formula (XII) and formula (XIII).

30. The method according to claim 29, in which the compounds of formula (III) are selected from the group consisting of random derivatives or derivatives with well-defined blocks having at least one of the following characteristics:
R″ is alkyl,
r″ is between 0 and 3 inclusive;
s is between 0 and 3 inclusive.

31. The method according to claim 27, in which the compounds of formula (III) are selected from the group consisting of:
2-(ethyl 4′-ylamino-α-cyanocinnamate)-4-(4-methoxyphenyl)-6-{[1,3,3,3-tetramethyl-1-[(trimethylsilyl)oxy]disiloxanyl]propyl-3-ylamino}-s-triazine,
2-(ethyl 4′-ylamino-α-cyanocinnamate)-4-(butyl 4′-ylaminobenzoate)-6-{[1,3,3,3-tetramethyl-1-[(trimethylsilyl)oxy]disiloxanyl]propyl-3-ylamino}-s-triazine,
2-(4′-ylamino-2-methanesulfonylacrylonitrile)-4-(butyl 4′-ylaminobenzoate)-6-{[1,3,3,3-tetramethyl-1-[(trimethylsilyl)oxy]disiloxanyl]propyl-3-ylamino}-s-triazine,
2-(ethyl 4′-ylamino-α-cyanocinnamate)-4-(butyl 4′-ylaminobenzoate)-6-(trimethylsilanylmethylylamino)-s-triazine,
2,4-bis(butyl 4′-diylaminobenzoate)-6-(2-ethylhexyl 4′-ylamino-α-cyanocinnamate)-s-triazine,
2,4,6-tris(isobutyl 4′-ylamino-α-cyanocinnamate)-s-triazine,
2,4,6-tris(2-ethylhexyl 4′-ylamino-α-cyanocinnamate)-s-triazine,
2,4-bis(2-ethylhexyl 4′-ylamino-α-cyanocinnamate)-6-{[1,3,3,3-tetramethyl-1-[(trimethylsilyl)oxy]disiloxanyl]propyl-3-ylamino}-s-triazine, and mixtures thereof.

32. The method according to claim 1, for protecting the complexion of the skin.

33. The method according to claim 1, for the natural antioxidant protection of the skin.

34. The method according to claim 1, for treating skin disorders caused by sunlight.

35. An aminoarylvinyl-s-triazine compound corresponding to formula (III) below: 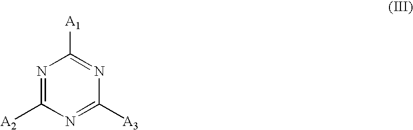
in which:
the groups A₁, A₂ and A₃, which may be identical or different, are selected from the group consisting of the groups of formulae (IV) to (VIII) below, it being understood that at least one group of formulae (IV) to (VII) is present: 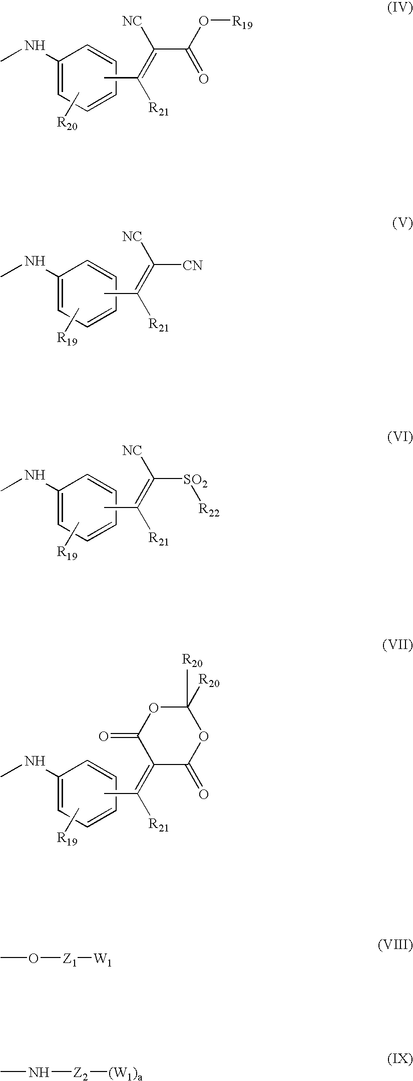
in which
R₂₀ represents hydrogen, C₁-C₄ alkyl radical or a C₁-C₄ alkoxy radical,
R₁₉ represents a linear or branched C₁-C₂₀ alkyl radical, a linear or branched C₂-C₂₀ hydroxyalkyl radical or a linear or branched C₁-C₂₀ alkoxy radical,
R₂₁ represents hydrogen, a methyl radical or a phenyl radical optionally substituted with a C₁-C₄ alkyl radical or a C₁-C₄ alkoxy radical,
R₂₂ represents a linear or branched C₁-C₂₀ alkyl radical or a phenyl radical optionally substituted with a C₁-C₄ alkyl radical or a C₁-C₄ alkoxy radical,
Z₁ represents a divalent radical providing the bond between —O— and —W₁,
Z₂ represents a divalent radical when a=1 and a trivalent radical when a=2, providing the bond between —NH— and —(W₁)a,
Z₁ and Z₂ possibly being C₁-C₁₂ alkylene, optionally substituted with one or more hydroxyl groups and possibly containing one or more oxygen atoms or one or more amino groups and optionally containing a double bond,
W₁ represents:
(i) either a silicone radical comprising at least one unit of formula (X) below: 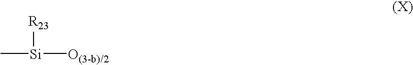
in which R₂₃ denotes a saturated or unsaturated, linear or branched C₁-C₃₀ hydrocarbon-based group; a C₁-C₈ halohydrocarbon group; a phenyl radical; a 3,3,3-trifluoropropyl radical or a trimethylsilyloxy group, and b is equal to 1 or 2, (ii) or a radical of formula (XI) below:
—SiR₂₄R₂₅R₂₆ (XI)
in which R₂₄, R₂₅ and R₂₆, which may be identical or different, are selected from the group consisting of linear or branched C₁-C₈ alkyl and alkenyl radicals; the groups A₁, A₂ and A₃ may also represent a UVB- and/or UVA-absorbing chromophore preferably selected from the group consisting of aryl groups substituted with hydroxyl groups, linear or branched C₁-C₂₀ alkyl groups and linear or branched C₁-C₂₀ alkoxy groups; aminobenzylidenecamphor groups; aminobenzotriazole groups; linear or branched aminobenzoate, aminobenzalmalonate, aminosalicylate, C₁-C₂₀ aminocinnamate or anthranilate ester groups;
with the proviso that:
the three groups A₁, A₂ and A₃ do not denote the same formula (IV),
when A₂ and A₃ denote a group of formula (IV), then A₁ does not denote a group of formula (VII) or (IX).

36. A compound according to claim 35, in which W is a silicone radical corresponding to one of the three formulae (XII) to (XIV) below: 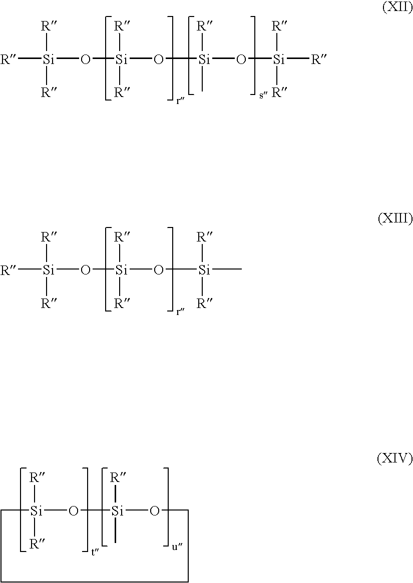
in which:
R″, which may be identical or different, are selected from the group consisting of linear or branched, saturated or unsaturated C₁-C₂₀ alkyl radicals, a phenyl radical and a 3,3,3-trifluoropropyl radical, at least 80% in numerical terms of the radicals R being methyl radicals,
r″ is an integer chosen between 0 and 50 inclusive,
s″ is an integer chosen between 0 and 20 inclusive,
u″ is an integer between 1 and 6 inclusive,
t″ is an integer between 0 and 10 inclusive,
t″+u″ is greater than or equal to 3.

37. A compound according to claim 36, in which W is a silicone radical corresponding to formula (XII) or to formula (XIII).

38. A compound according to claim 37, selected from the group consisting of random derivatives or derivatives with well-defined blocks having at least one and even more preferably all of the following characteristics:
R″ is alkyl,
r″ is between 0 and 3 inclusive; s is between 0 and 3 inclusive.

39. A compound according to claim 35, selected from the group consisting of:
2,4-bis(butyl 4′-diylaminobenzoate)-6-(2-ethylhexyl 4′-ylamino-α-cyanocinnamate)-s-triazine; and
2-(4′-ylamino-2-methanesulfonylacrylonitrile)-4-(butyl 4′-ylaminobenzoate)-6-{[1,3,3,3-tetramethyl-1-[(trimethylsilyl)oxy]disiloxanyl]propyl-3-ylamino}-s-triazine.

40. A composition comprising a physiologically acceptable medium and at least one aminoarylvinyl-s-triazine compound of formula (III) as claimed in claim 35.

41. The composition according to claim 40, in which the compound(s) of formula (m) are present in a concentrations of from 0. 1% to 15% by weight relative to the total weight of the composition.

42. The composition according to claim 40, further comprising at least one UVA-active and/or UVB-active organic photoprotective agent and/or at least one UVA-active and/or UVB-active mineral photoprotective agent.

43. The composition according to claim 42, comprising at least one organic photoprotective agent are selected from the group consisting of anthranilates; cinnamic derivatives; dibenzoylmethane derivatives; salicylic derivatives; camphor derivatives; triazine derivatives; benzophenone derivatives; β,β′-diphenylacrylate derivatives; benzotriazole derivatives; benzalmalonate derivatives; benzimidazole derivatives; imidazolines; bis-benzazolyl derivatives; p-aminobenzoic acid (PABA) derivatives; methylenebis(hydroxyphenylbenzotriazole) derivatives; benzoxazole derivatives; screening polymers and screening silicones; dimers derived from α-alkylstyrene; 4,4-diarylbutadienes, and mixtures thereof.

44. A composition according to claim 43, in which the organic photoprotective agents are selected from the group consisting of the following compounds:
Ethylhexyl salicylate,
Butyl methoxydibenzoylmethane,
Ethylhexyl methoxycinnamate,
Octocrylene,
Phenylbenzimidazolesulfonic acid,
Terephthalylidenedicamphorsulfonic acid,
Benzophenone-3,
Benzophenone-4,
Benzophenone-5,
n-Hexyl 2-(4-diethylamino-2-hydroxybenzoyl)benzoate,
4-Methylbenzylidenecamphor,
Disodium phenyldibenzimidazoletetrasulfonate,
Anisotriazine,
Ethylhexyltriazone,
Diethylhexylbutamidotriazone,
2,4,6-Tris(diisobutyl 4′-aminobenzalmalonate)-s-triazine,
Methylenebis(benzotriazolyl)tetramethylbutylphenol,
Drometrizole trisiloxane,
Polysilicone-15,
1,1-Dicarboxy(2,2′-dimethylpropyl)-4,4-diphenylbutadiene,
2,4-Bis[5-1(dimethylpropyl)benzoxazol-2-yl(4-phenyl)imino]-6-(2-ethylhexyl)imino-1,3,5-triazine, and mixtures thereof.

45. A composition according to claim 42, comprising a mineral photoprotective agent selected from the group consisting of coated or uncoated metal oxide pigments or nanopigments, and mixtures thereof.

46. A composition according to claim 45, in which the mineral photoprotective agents are nanopigments of titanium oxide, iron oxide, zinc oxide, zirconium oxide or cerium oxide, which are coated or uncoated.

47. A composition according to claims 42, in which the photoprotective agents are present in proportions ranging from 0.01% to 20% by weight relative to the total weight of the composition.

48. A composition according to claims 42, further comprising an agent for artificially tanning and/or browning the skin.

49. A composition according to claims 42, further comprising at least one cosmetic adjuvant selected from the group consisting of fatty substances, organic solvents, ionic or nonionic, hydrophilic or lipophilic thickeners, softeners, humectants, opacifiers, stabilizers, emollients, silicones, antifoams, fragrances, preserving agents, anionic, cationic, nonionic, zwitterionic or amphoteric surfactants, active agents, fillers, polymers, propellants, acidifying or basifying agents or any other ingredient usually used in cosmetics and/or dermatology.
